(19) United States
(12) Patent Application Publication
(10) Pub. No.: US 2020/0399196 A1
(43) Pub. Date: Dec. 24, 2020

(54) Hydrogen Bond Directed Photocatalytic Hydrodefluorination and Methods of Use Thereof

(71) Applicant: The Board of Regents for Oklahoma State University, Stillwater, OK (US)

(72) Inventor: Jimmie Dean Weaver, Stillwater, OK (US)

(21) Appl. No.: 16/494,010

(22) PCT Filed: Apr. 3, 2018
(86) PCT No.: PCT/US18/25894

§ 371 Date: Sep. 13, 2019

Related U.S. Application Data
(60) Provisional application No. 62/480,748, filed on Apr. 3, 2017.

(51) Int. Cl. C07C 51/06; B01J 31/02; B01J 35/00; C07C 253/30; C07D 235/30

(52) U.S. Cl. CPC C07C 51/06; B01J 31/0237; B01J 35/004; C07C 57/58; C07D 235/30; B01J 2231/64; B01J 2531/827; C07C 253/30

(57) ABSTRACT

Methods of synthesizing compounds comprising fluorinated aryl groups are disclosed, wherein said methods utilize hydrogen bond directed photocatalytic hydrodefluorination.

### CROSS REFERENCE TO RELATED APPLICATIONS/INCORPORATION BY REFERENCE STATEMENT

This application claims benefit under 35 USC § 119(e) of U.S. Provisional Application No. 62/480,748, filed Apr. 3, 2017. The entire contents of the above-referenced application are hereby expressly incorporated herein by reference.

### STATEMENT REGARDING FEDERALLY SPONSORED RESEARCH OR DEVELOPMENT

This invention was made with U.S. Government support under NIH Grant No. 1R01GM115697-01 awarded by the Department of Health and Human Services. The Government has certain rights in this invention.

### BACKGROUND

Multifluorinated arenes are proving to be an important class of molecules for materials, pharmaceuticals, agrochemicals, and catalysts. However, in sharp contrast to the utility of multifluorinated arenes is the ease of their syntheses, which often are surprisingly tedious to access. This difficulty stems from both the difficulty in forming C-F bonds as well as the regioselectivity. While recent developments in methodology have made accessing mono-fluoroarenes significantly more tractable, they have not solved the problem of multifluorinated arenes. This is because of the methods either rely on converting pre-installed functional groups (i.e., halides or metals) or the directed substitution of C-H bonds. However, in the case of multifluorinated arenes, the selective installation of the necessary functional groups simply shifts the difficult steps to earlier in the synthesis. While directed functionalization avoids this issue, it is generally limited to ortho functionalization of the directing group.

In contrast, perfluorination of the arenes, in which a C—F bond has replaced every C—H bond, is readily accomplished on a commercial scale and completely removes the problem of difficult installation of the fluorine. The challenge then becomes selective C—F functionalization/reduction. If developed successfully, the potential value of this approach is enormous, since each fluorine has the potential to be functionalized. The possibilities have been recognized for some time and have been championed by others (Lentz et al., 2013; Ahrens et al., 2015; Weaver et al., 2014; Kiplinger et al., 1994; and Amii et al., 2009). However, there are many challenges associated with the C—F functionalization approach. For instance, oxidative addition into the short and strong C—F bond is challenging (for C—F of C₆F₆, 1.3250 Å (Den et al., 2014), 145 kcal/mol (Konovalov et al., 2000)). Furthermore, if successful, many catalysts form strong metal-F bonds that lead to sluggish turnover (Lentz et al., 2013).

In 2014, Senaweera et al. (2014) introduced the photocatalytic-HDF (hydrodefluorination) reaction, which circumvented some of the aforementioned issues. Specifically, Senaweera et al. (2014) showed that when an Ir-based photocatalyst, tertiary amine, and visible light were combined, facile hydrodefluorination occurred. At the basic level, the reaction is believed to proceed through selective single electron reduction of perfluoroarenes (A, FIG. 2), to give a perfluoroaryl radical anion (B). The radical anion then undergoes C—F fragmentation to neatly generate a perfluoroaryl radical (C) which underwent selective hydrogen atom transfer from the amine radical cation or amine to give the HDF product (D)(Senaweera et al., 2014). More recently, Xie et al. (2016) have shown the photocatalytic electron transfer/fluoride fragmentation strategy can be used to engage difluorostyrenes, and McTeague et al. (2016) have shown that it can be used to activate even inert SF₆. These diverse results attest to the generality of the photocatalytic reductive fragmentation strategy.

Returning to the problem of multifluorinated arenes, the inventor has further demonstrated that the perfluoroaryl radical is a powerful intermediate for the functionalization of perfluoroarenes which can give rise to C—F alkylation (Singh et al., 2015), arylation (Senaweera et al., 2016), and alkenylation (Singh et al., 2016). While the perfluoroaryl radical has proved competent for cross-coupling, the inherent limitation is the regioselectivity of the C—F fragmentation event, which, while generally regioselective, is dictated by the electronics of each substrate, and therefore represents a major limitation to the photocatalytic C—F functionalization strategy. Therefore, in order for the field to advance, strategies are needed which provide alternative C—F fragmentation regioselectivities.

### BRIEF DESCRIPTION OF THE DRAWINGS

FIG. 1 contains examples of commercially valuable multifluorinated arenes, a typical prior art synthesis of multifluorinated arenes, and a comparison of the strategy utilized in the methods of the present disclosure versus other modern strategies to access fluorinated arenes.

FIG. 2 contains an exemplary working mechanism of photocatalytic-HDF.

FIG. 3 contains non-limiting examples of some initial results in the directed Photo-HDF method according to one embodiment.

FIG. 4 contains an illustration of an embodiment which utilizes directly attached directing groups.

FIG. 5 contains a non-limiting example of exploration of other directing groups for tetrafluoropyridine.

FIG. 6 illustrates a non-limiting example of exploration of other polyfluoroarenes in the directed Photo-HDF reaction.

FIG. 7 contains a non-limiting example of exploration of 6-membered hydrogen bonding substrates.

FIG. 8 illustrates a non-limiting example of directed HDF with sterically hindered amides.

FIG. 9 contains a non-limiting example of exploration of 6-membered hydrogen bonding substrates.

FIG. 10 contains a non-limiting example of exploration of 7-membered hydrogen bonding substrates.

FIG. 11 contains a non-limiting example of exploration of a benzoate motif.

FIG. 12 contains a non-limiting exemplary illustration of syntheses for a key fluorinated starting material for JANUVIA® (sitagliptin, Merck & Co., Inc., Kenilworth, N.J.) by a traditional synthesis method (A) and by a directed photocatalytic-HDF approach of the present disclosure.

FIG. 13 contains a pseudo-Hammett plot of the directed photocatalytic-HDF reaction of tetrafluoropyridine. The average rate at ca. 20% conversion is plotted against the sigma-para values (McDaniel et al., 1958).

FIG. 14 illustrates a linear free energy relationship of the HDF regioselectivity as a function of σₚ⁻ Hammett values (Hansch et al., 1991) in the methyl benzoate series with a remote directing group.

FIG. 15 contains (A) a scheme for directed photo-HDF when the directing group formed an 8-membered hydrogen bonding cycle (A), and (B) a scheme for probing the robustness of the directed photocatalytic-HDF with respect to amount of photocatalyst used.

FIG. 16 contains a scheme for second generation synthesis of 2,4,5-trifluorophenylacetic acid.

FIG. 17 illustrates synthesis of 3-chloro-2,4,5,6-tetrafluorobenzoyl chloride.

FIG. 18 illustrates synthesis of 1-(3-chloro-2,4,5,6-tetrafluorophenyl)-2-diazoethan-1-one.

FIG. 19 illustrates synthesis of 2-(3-chloro-2,4,5,6-tetrafluorophenyl)-N-(4-cyanophenyl)acetamide.

FIG. 20 illustrates synthesis of N-(4-cyanophenyl)-2-(2,4,5-trifluorophenyl)acetamide.

FIG. 21 illustrates synthesis of 2,4,5-trifluorophenylacetic acid.

FIG. 22 contains a scheme for first generation synthesis of 2,4,5-trifluorophenylacetic acid.

FIG. 23 illustrates synthesis of 3-chloro-2,4,5,6-tetrafluorobenzoyl chloride.

FIG. 24 illustrates synthesis of 3-chloro-N-(4-cyanophenyl)-2,4,5,6-tetrafluorobenzamide.

FIG. 25 illustrates synthesis of 2,4,5,-trifluorobenzoic acid.

FIG. 26 illustrates synthesis of 2,4,5-trifluorobenzoic acid.

FIG. 27 illustrates synthesis of 2,4,5-trifluorobenzoyl chloride.

FIG. 28 illustrates synthesis of 2-diazo-1-(2,4,5-trifluorophenyl)ethan-1-one.

FIG. 29 illustrates synthesis of 2-(2,4,5-trifluorophenyl)acetic acid.

FIG. 30 illustrates synthesis of JANUVIA® (sitagliptin, Merck & Co., Inc., Kenilworth, N.J.) acid by the traditional prior art method (upper scheme) and by the methods of the present disclosure (lower scheme).

FIG. 31 illustrates synthesis of DIFLUCAN® (fluconazole, Pfizer Consumer Healthcare, Mississauga, ON) by the traditional prior art method (lower scheme) and by the methods of the present disclosure (upper scheme).

FIG. 32 illustrates synthesis of melperone by the traditional prior art method (lower scheme) and by the methods of the present disclosure (upper scheme).

FIG. 33 illustrates synthesis of blonanserin by the traditional prior art method (lower scheme) and by the methods of the present disclosure (upper scheme).

FIG. 34 illustrates synthesis of carbonic anhydrase inhibitor by the traditional prior art method (upper scheme) and by the methods of the present disclosure (lower scheme).

FIG. 35 illustrates synthesis of rufinamide by the traditional prior art method (lower scheme) and by the methods of the present disclosure (upper scheme).

FIG. 36 illustrates synthesis of elvitegravir by the traditional prior art method (lower scheme) and by the methods of the present disclosure (upper scheme).

FIG. 37 illustrates synthesis of dolutegravir by the traditional prior art method (lower scheme) and by the methods of the present disclosure (upper scheme).

FIG. 38 illustrates synthesis of darapladib by the traditional prior art method (lower scheme) and by the methods of the present disclosure (upper scheme).

FIG. 39 illustrates synthesis of oxo-ezogabine analog by the traditional prior art method (lower scheme) and by the methods of the present disclosure (upper scheme).

FIG. 40 illustrates individual steps in the synthesis of fluconazole.

FIG. 41 illustrates individual steps in the synthesis of melperone.

FIG. 42 illustrates individual steps in the synthesis of blonanserin.

FIG. 43 illustrates individual steps in the synthesis of carbonic anhydrase inhibitor.

FIG. 44 illustrates individual steps in the synthesis of rufinamide.

FIG. 45 illustrates individual steps in the synthesis of dolutegravir intermediates.

FIG. 46 illustrates individual steps in the synthesis of darapladib intermediate.

FIG. 47 illustrates individual steps in the synthesis of oxo-ezogabine analog.

FIG. 48 illustrates individual steps in the synthesis of ezogabine.

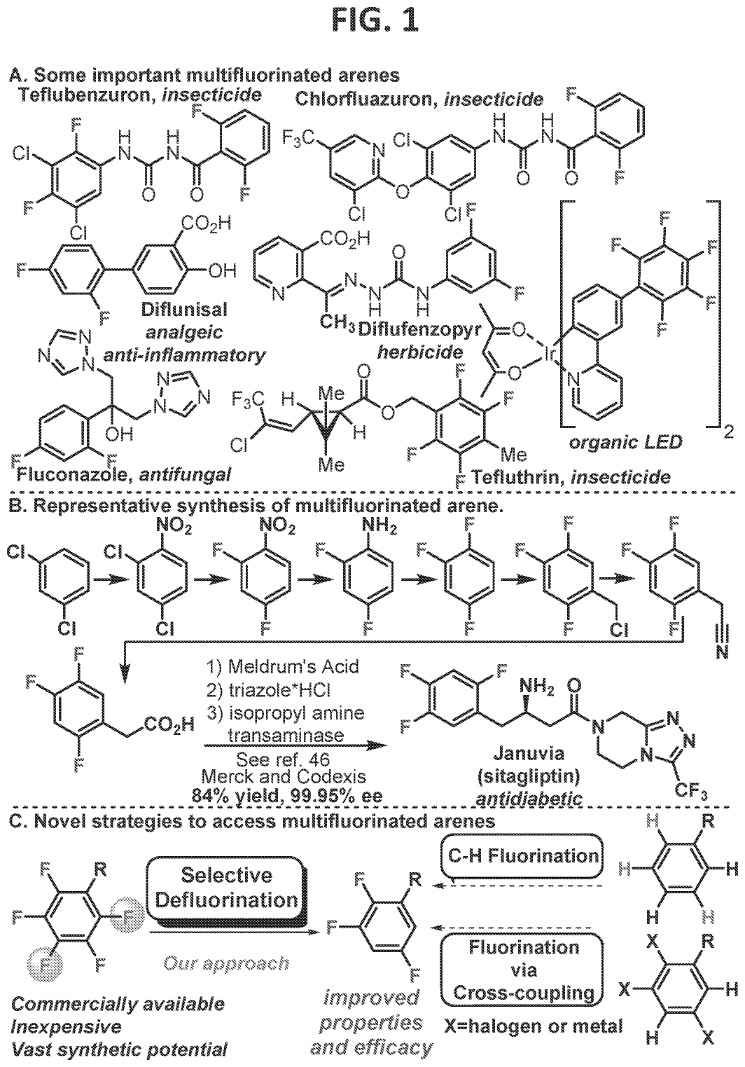

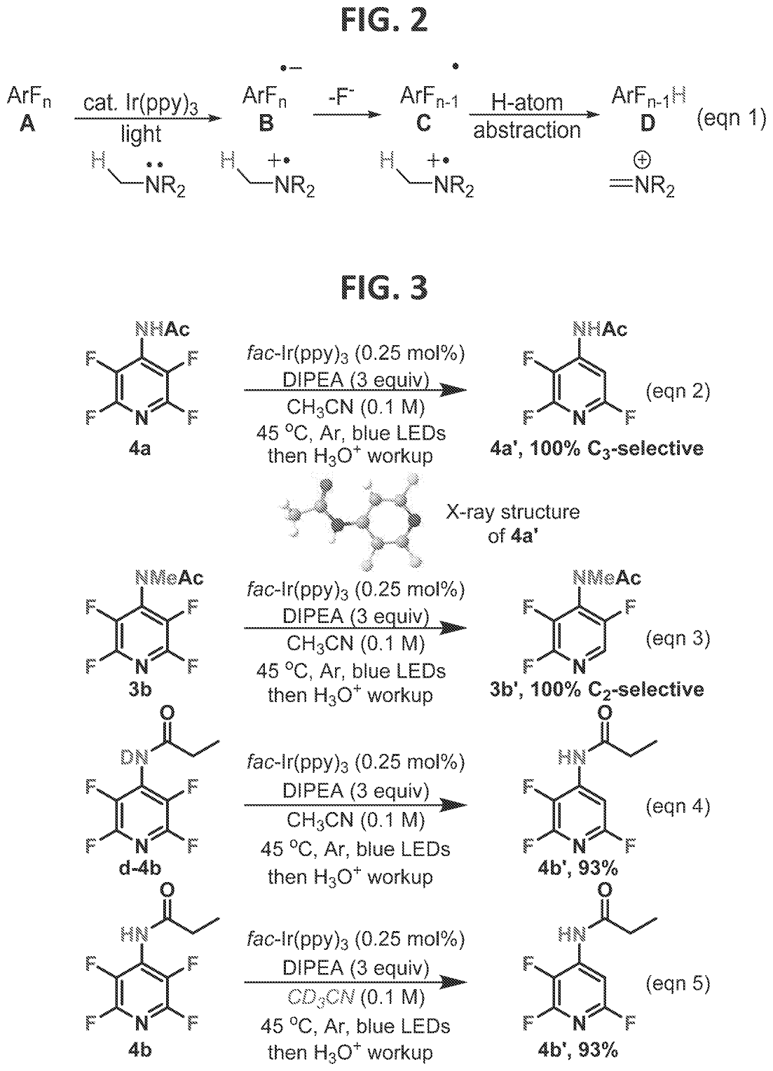

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, “substantially pure” means an object species is the predominant species present (i.e., on a molar basis it is more abundant than any other individual species in the composition), and a substantially purified fraction is a composition wherein the object species comprises at least about 50 percent (on a molar basis) of all macromolecular species present. Generally, a substantially pure composition will comprise more than about 80 percent of all macromolecular species present in the composition, such as (but not limited to) more than about 85%, 90%, 95%, and 99%. In a particular (but non-limiting) embodiment, the object species is purified to essential homogeneity (contaminant species cannot be detected in the composition by conventional detection methods), wherein the composition consists essentially of a single macromolecular species.

Turning now to the inventive concept(s), certain non-limiting embodiments of the present disclosure are directed to a method of synthesizing a compound comprising a fluorinated aryl group utilizing hydrogen bond directed photocatalytic hydrodefluorination. The method includes the steps of: reacting a perhalogenated arene with a directing group to install the directing group on the perhalogated arene, resulting in a compound comprising a perhalogenated aryl group directly attached to a directing group comprising an acidic proton; and reacting the compound comprising the perhalogenated aryl group and the directing group with a photocatalyst to regioselectively remove at least one halogen therefrom, thereby providing the compound comprising a fluorinated aryl group.

Any perhalogenated arene known in the art or otherwise contemplated herein may be utilized in accordance with the present disclosure. Non-limiting examples of perhalogenated arenes that may be utilized in accordance with the present disclosure include hexahalogenated benzene, pentahalogenated benzoate, methyl pentahalogenated benzoate, pentahalogenated toluene, pentahalogenated benzoic acid, pentahalogenated benzonitrile, pentahalogenated aniline, pentahalogenated benzamide, a pentahalogenated benzamido group, a pentahalogenated benzoyl group, and tetrahalogenated pyridine.

The term “perhalogenated arene” as utilized herein represents any arene group in which all of the hydrogens of the arene ring have been replaced with a halogen. In certain non-limiting embodiments, all of the hydrogens of the arene ring are replaced with a single halogen (such as, but not limited to, fluorine (i.e., a perfluoroarene)). In other non-limiting embodiments, multiple halogens may be present (i.e., a combination of fluorine with one or more of chlorine, bromine, and/or iodine).

In a particular (but non-limiting) embodiment, the perhalogated arene is a perfluorinated arene. Non-limiting examples of perfluorinated arenes that may be utilized in accordance with the present disclosure include hexafluorobenzene, pentafluorobenzoate, methyl pentafluorobenzoate, pentafluorotoluene, pentaflourobenzoic acid, pentafluorobenzonitrile, pentafluoroaniline, pentafluorobenzamide, a pentafluorobenzamido group, a pentafluorobenzoyl group, and tetrafluoropyridine.

In another particular (but non-limiting) embodiment, the perhalogenated arene may include a combination of one or more fluorines with one or more chlorines.

In another particular (but non-limiting) embodiment, the perhalogenated arene is selected from the group consisting of a perhalogenated benzoyl chloride, perhalogenated benzenecarboxylic acid, perhalogenated phenyl(cyanophenyl)acetamide, and methyl perhalogenated benzoate.

One of the advantages of each of the methods of the present disclosure is the ability to regioselectively remove halogens at particular position(s) of the arene ring. One of the major limitations to the prior art photocatalytic C—F functionalization strategies are that they are dictated by the electronics of each substrate, and as such, do not allow for selective removal of the halogens. In particular, halogens at the para-position are typically removed first. In contrast, the methods of the present disclosure allow for regioselective removal of at least one halogen from an ortho-position and/or a meta-position, as well as regioselective removal of at least one halogen from a para-position. In a particular (but non-limiting) example, at least one halogen is regioselectively removed from an ortho-position, and at least one halogen is regioselectively removed from a meta-position.

Any directing groups known in the art or otherwise contemplated herein that are capable of functioning in accordance with the present disclosure can be utilized in accordance with each of the methods described herein above and herein below. The only requirement for a directing group utilized in accordance with the methods of the present disclosure is that the directing group includes an acidic protein that causes a change in regioselectivity; in this manner, C—F bonds (or other C-halogen bonds) at locations which do not typically undergo fragmentation can be enticed to do so by the strategic positioning of the acidic proton of the directing group.

Likewise, any acidic protons known in the art or otherwise contemplated herein that are capable of functioning in accordance with the present disclosure may be utilized as the acidic proton of the directing group. In one particular (but non-limiting) embodiment, the acidic proton is an acidic NH.

Non-limiting examples of directing groups that can be utilized in accordance with the methods of the present disclosure include an amine group (such as, but not limited to, an arylamine group), a nitrile group (such as, but not limited to, a benzonitrile, an aminobenzonitrile, or an acetonitrile group), a pyrazole group (such as, but not limited to, a benzopyrazole group), a pyridine group, an azide group, an aniline group, and a benzoimidazolone group (such as, but not limited to, an aminodihydrobenzoimidazolone) group.

In a particular (but non-limiting) embodiment, the perhalogenated arene is a perhalogenated benzoyl chloride (such as, but not limited to, 3-chloro-tetrafluorobenzoyl chloride), the directing group is an aminobenzonitrile, and at least the halogens at a 3-position and a 6-position are removed. For example (but not by way of limitation), the chlorine at the 3-position and a fluorine at the 6-position are removed (such as, but not limited to, in the synthesis of sitagliptin, as described in detail in the Examples).

In certain particular (but non-limiting) embodiments, the perhalogated arene is a perhalogenated benzoic acid. For example, but not by of limitation, the perhalogenated arene may be 3,5-dichloro-2,4,6-trifluorobenzoic acid (such as, but not limited to, in the synthesis of fluconazole, elvitegravir, dolutegravir, darapladib, and oxo-ezogabine analog, as described in detail in the Examples). For example (but not by way of limitation), the directing group utilized with this perhalogenated arene can be an aminobenzonitrile (such as, but not limited to, in the synthesis of Fluconazole, Elvitegravir, Dolutegravir, and Darapladib, as described in detail in the Examples), and at least the two halogens at meta-positions and one or both halogens at the ortho-positions are removed. For example, but not by way of limitation, the two chlorines and a fluorine at an ortho-position may be removed (such as, but not limited to, in the synthesis of Fluconazole, Elvitegravir, and Dolutegravir, as described in detail in the Examples). In an alternative (but non-limiting) example, the two chlorines and each of the fluorines at ortho-positions are removed (such as, but not limited to, in the synthesis of Darapladib, as described in detail in the Examples). In another particular (but non-limiting) embodiment, the directing group is an aminodihydrobenzoimidazolone, and at least the two halogens at meta-positions and the two halogens at the ortho-positions are removed; for example (but not by way of limitation), the two chlorines and each of the fluorines at ortho-positions are removed (such as, but not limited to, in the synthesis of oxo-ezogabine analog, as described in detail in the Examples).

Certain non-limiting embodiments of the present disclosure are directed to a method of synthesizing a compound comprising a fluorinated aryl group utilizing hydrogen bond directed photocatalytic hydrodefluorination, in which the method comprising the steps of: reacting a perhalogenated benzoyl chloride (such as, but not limited to, 3-chloro-tetrafluorobenzoyl chloride) with an aminobenzonitrile, resulting in a compound comprising a perhalogenated aryl group directly attached to a directing group comprising an acidic proton; and reacting the compound comprising the perhalogenated aryl group and the directing group with a photocatalyst to regioselectively remove at least one halogen therefrom, thereby providing the compound comprising a fluorinated aryl group, and wherein at least one halogen is regioselectively removed from at least one of an ortho-position and a meta-position. In a particular (but non-limiting) embodiment, a chlorine at the 3-position and a fluorine at the 6-position are removed. The method may proceed as disclosed, for example but not by way of limitation, for the synthesis of sitagliptin, as described in the detail in the Examples.

Certain non-limiting embodiments of the present disclosure are further directed to a method of synthesizing a compound comprising a fluorinated aryl group utilizing hydrogen bond directed photocatalytic hydrodefluorination, in which the method comprises the steps of: reacting perhalogenated benzoic acid (such as, but not limited to, 3,5-dichloro-2,4,6-trifluorobenzoic acid) with a directing group, resulting in a compound comprising a perhalogenated aryl group directly attached to a directing group comprising an acidic proton; and reacting the compound comprising the perhalogenated aryl group and the directing group with a photocatalyst to regioselectively remove at least one halogen therefrom, thereby providing the compound comprising a fluorinated aryl group, and wherein at least one halogen is regioselectively removed from at least one of an ortho-position and a meta-position. The method may proceed as disclosed, for example but not by way of limitation, for the synthesis of any of fluconazole, elvitegravir, dolutegravir, darapladib, and oxo-ezogabine analog, as described in detail in the Examples.

In certain particular (but non-limiting) embodiments, the directing group utilized with this perhalogenated arene can be an aminobenzonitrile (such as, but not limited to, in the synthesis of Fluconazole, Elvitegravir, Dolutegravir, and/or Darapladib, as described in detail in the Examples), and the halogens removed may be as follows: (1) the two chlorines and a fluorine at an ortho-position are removed (such as, but not limited to, in the synthesis of Fluconazole, Elvitegravir, and/or Dolutegravir, as described in detail in the Examples); or (2) the two chlorines and each of the fluorines at ortho-positions are removed (such as, but not limited to, in the synthesis of Darapladib, as described in detail in the Examples). In another non-limiting example, the directing group is an aminodihydrobenzoimidazolone, and the two chlorines and each of the fluorines at ortho-positions are removed (such as, but not limited to, in the synthesis of oxo-ezogabine analog, as described in detail in the Examples).

Certain non-limiting embodiments of the present disclosure are directed to a method of synthesizing a compound comprising a fluorinated aryl group utilizing hydrogen bond directed photocatalytic hydrodefluorination, in which the method comprises the steps of: reacting perhalogenated phenylacetamide (such as, but not limited to, 2-(3,5-dichloro-2,4,6-trifluorophenyl)-N-(4-cyanophenyl)acetamide) with a photocatalyst to regioselectively remove at least one halogen from at least one of an ortho-position and a meta-position, thereby producing a fluorinated aryl group; and reacting the fluorinated aryl group with a directing group, thereby providing the compound comprising a fluorinated aryl group and a directing group. The method may proceed as disclosed, for example but not by way of limitation, for the synthesis of Melperone and/or Blonanserin, as described in detail in the Examples.

In certain non-limiting embodiments, all halogens except a fluorine at a para-position are removed. In a particular (but non-limiting) embodiment, the directing group is a pyridine group (such as, but not limited to, in the synthesis of Melperone, as described in detail in the Examples). In another particular (but non-limiting) embodiment, the directing group is an acetonitrile group (such as, but not limited to, in the synthesis of Blonanserin, as described in detail in the Examples).

Certain non-limiting embodiments of the present disclosure are directed to a method of synthesizing a compound comprising a fluorinated aryl group utilizing hydrogen bond directed photocatalytic hydrodefluorination, in which the method comprises the steps of: reacting methyl perhalogenated benzoate (such as, but not limited to, methyl 3,5-dichloro-2,4,6-trifluorobenzoate) with a photocatalyst to regioselectively remove at least one halogen from at least one of an ortho-position and a meta-position, thereby producing a fluorinated aryl group; and reacting the fluorinated aryl group with a directing group, thereby providing the compound comprising a fluorinated aryl group and a directing group. The method may proceed as disclosed, for example but not by way of limitation, for the synthesis of Rufinamide, as described in detail in the Examples.

In a particular (but non-limiting) example, the two chlorines and a fluorine at the para-position are removed, and wherein the directing group is an azide group (such as, but not limited to, in the synthesis of Rufinamide, as described in detail in the Examples).

Each of the methods described or otherwise contemplated herein can produce the compound comprising a fluorinated aryl group with any level of yield and without requiring chromatography steps. For example (but not by way of limitation), the compound comprising a fluorinated aryl group can be synthesized with a yield of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, and at least about 99% without requiring any chromatography steps. In addition, the scope of the presently disclosure also includes the production of the compound comprising a fluorinated aryl group at any percent yield that falls within any range formed from the combination of two values listed above (for example, a range of from about 10% to about 99%, a range of from about 30% to about 98%, a range of from about 50% to about 97%, a range of from about 60% to about 96%, a range of from about 70% to about 95%, etc.).

Any photocatalysts known in the art or otherwise contemplated herein for use with a method of hydrodefluorination can be utilized in accordance with any of the methods of the present disclosure. For example (but not by way of limitation), the photocatalyst can be Ir(ppy)₃. In addition, non-limiting examples of other photocatalysts that may be utilized in accordance with the present disclosure include those disclosed in International Application No. PCT/US18/21112, filed Mar. 6, 2018 (the entire contents of which are hereby expressly incorporated herein by reference).

Each of the methods disclosed or otherwise contemplated herein may be performed in batch reactions or via continuous flow methodologies (as demonstrated for substrate controlled photo-HDF methods in, for example, Senaweera et al., 2014). In addition, each of the methods disclosed or otherwise contemplated herein may be performed as a one-pot synthesis method (i.e., multiple reactions performed in a single reactor) or may be performed in multiple reactors.

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

### Example 1—Hydrogen Bond Directed Photocatalytic HDF: Overcoming Electronic Control

As stated above in the Background section, with respect to multifluorinated arenes, the inventor has further demonstrated that the perfluoroaryl radical is a powerful intermediate for the functionalization of perfluoroarenes which can elicit C—F alkylation (Singh et al., 2015), arylation (Senaweera et al., 2016), and alkenylation (Singh et al., 2016). While the perfluoroaryl radical has proven competent for cross-coupling, the inherent limitation is the regioselectivity of the C—F fragmentation event. Despite this fragmentation being generally regioselective, it is dictated by the intrinsic electronics of each substrate, and therefore until now has been an obstinate limitation to the photocatalytic C—F functionalization strategy. Therefore, in order for the field to advance, strategies are needed which provide alternative C—F fragmentation regioselectivities from the same motifs. With this goal in mind, the rudiments of the C—F regioselectivity in the photocatalytic-HDF and C—F functionalization reactions, and specifically, how they might be subverted, were contemplated.

The structure of the perfluoroaryl radical anion of hexafluorobenzene is known to be nonplanar, in which the C—F bonds are contorted out of the plane of the ring (Shchegoleva et al., 1999). In fact, the fragmentation process is greatly accelerated by the nonplanarity, because it allows for significant mixing of the n* and the C-F σ* orbitals, effectively lifting the otherwise symmetry forbidden intramolecular electron transfer. Given the importance of both the directionality and shape of the C—F σ* orbital for overlap with the arene n* orbital, it stands to reason that perturbation of either the length or direction of σ* orbitals of the C—F bonds will thus influence the likelihood of the bond's fragmentation from the radical anion which is formed upon electron addition.

Thus, it was postulated that the C—F bonds at locations which do not typically undergo fragmentation could be enticed to do so by the strategic positioning of an acidic proton. Literature suggests that while hydrogen bonding with organofluorines is a weak interaction (Dalvit et al., 2014; Schneider, 2012; and Champagne et al., 2015), hydrogen bonding with the fluoride ion is strong (Hossain et al., 2012; Mason et al., 2000; and Hossain et al., 2002). Unfortunately, this provides little insight with respect to the ability of an organofluorine radical anion, which possesses an intermediate amount of negative charge, to engage in hydrogen bonding. Additionally, since solvation of fluoride is a highly exothermic process (Zhan et al., 2004), it was expected that the fluoride fragmentation event would become more exothermic because the newly formed fluoride would already be engaged in a hydrogen bond (Baird et al., 2017).

Results and Discussion of Example 1

This Example was initiated with N-acetylated tetrafluoropyridine, 4a, which was subjected to standard photo-HDF conditions, which consisted of catalytic amounts of (fac-Ir(ppy)₃) (tris[2-phenylpyridinato-C²,N]iridium(III)), and three equivalents of DIPEA (diisopropylethylamine) (FIG. 3, eqn 2). Smooth and efficient HDF with complete C₃ selectivity was observed, as confirmed by an X-ray structure of the product, in contrast to the electronically favored position, C₂. When the nitrogen of the substrate was methylated (3b), removing the acidic NH, and subjected to the same reaction conditions, it underwent photo-HDF exclusively at C₂, though notably more slowly (eqn 3). Methylation of 4a′ allowed direct comparison to 3b′ and confirmed the correlation of regioselectivity to the presence or absence of the acidic proton.

Deuterium labeling studies demonstrate that neither the acidic proton (eqn 4) nor the solvent (eqn 5) was serving as the H-atom source. This further demonstrates that the importance of NH is to undergo hydrogen bonding with the fluoride, rather than serving as an H-atom source. Previously, the amine (or its radical cation) was shown to be the source of the H-atom in the phot-HDF (Senaweera et al., 2014), and these results are consistent with the previous findings.

This result is consistent with the idea that manipulation of the C—F a* orbital is a viable strategy for obtaining alternative regioselectivity in the reductive fragmentation. This is consistent with Laev et al.'s (2007) observations that zinc ions accelerated the dissolving metal-HDF with N-acetylated polyfluoroamines, in which it was proposed that zinc ion coordination of the acetyl group and the ortho fluorine both accelerate the reaction and alter the regioselectivity. Given both the mild reaction conditions of the photo-HDF and the ubiquity of acidic protons within polyfluorinated molecules of interest, it was surmised that this chemistry has significant potential to alter how multifluorinated arenes are synthesized. Thus, the reaction was studied to try to understand the limitations and requirements.

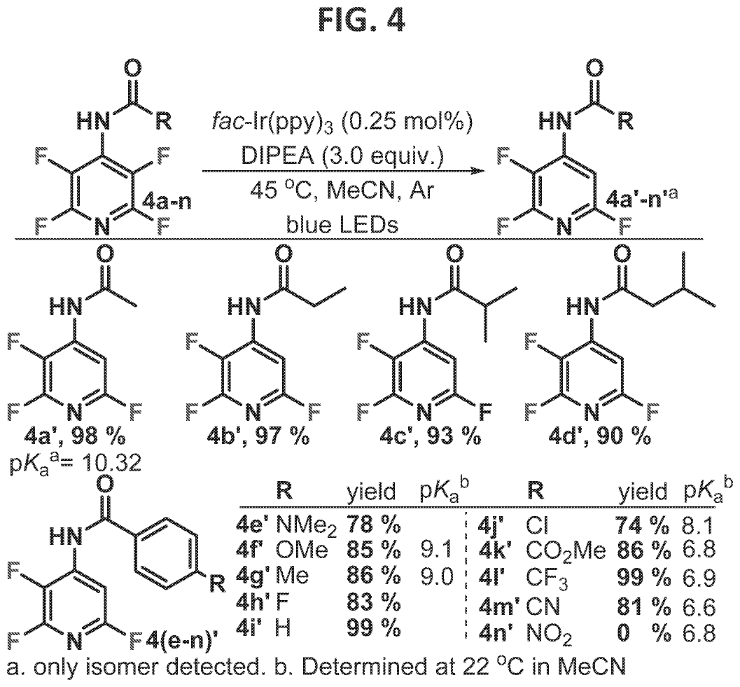

Substrates such as 4 are rapidly synthesized from inexpensive pentafluoropyridine in two steps, and thus the scope of directing groups in which the acidic proton is directly attached to the tetrafluoropyridine ring was initially explored (FIG. 4). The steric demand of the acetyl group has little impact on the isolated yield (4a′-4d′), which uniformly gave high yields. It was found that benzoyl amides also generally served to direct the HDF event. The HDF event took place smoothly for both electron rich (4e′-g′), neutral (4h′-i′), and electron poor amides (4j′-n′). In the case of the nitro-substituted benzoyl amide, the reaction failed to take place. This likely resulted from competitive quenching of the photocatalyst by the nitroarene motif, which has previously been observed (Prier et al., 2013).

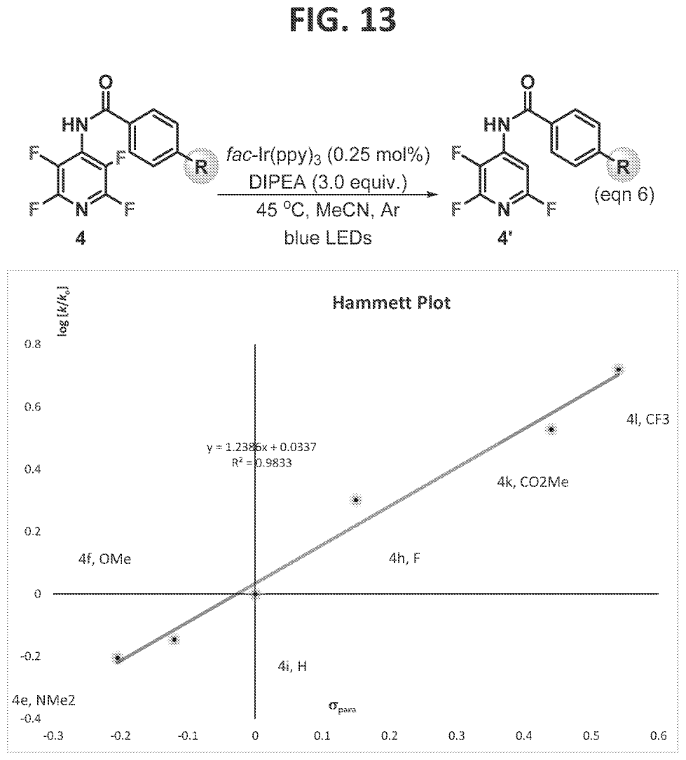

At low conversion (ca. 20%), an average rate was determined, and a pseudo-Hammett plot was constructed (FIG. 13). A positive p value (1.24) is consistent with a buildup of negative charge during the rate determining step (RDS). This result indicates that the RDS is fluoride fragmentation, in which partial proton transfer to a fluoride occurs, and explains the prominent role the acidity of the hydrogen bond donor plays in the reaction outcome.

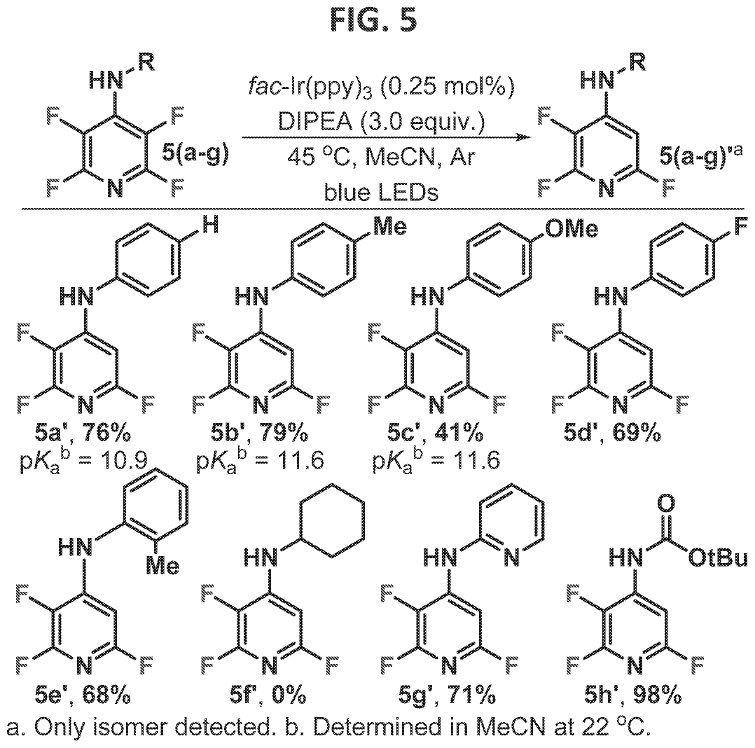

The type of H-bond donors which were competent at facilitating the directed photo-HDF reaction was explored next. In general, only N—H groups were found to be ideally suitable, though others were explored and are discussed in Example 2. Given the prevalence of N—H bonds in pharmaceuticals and other polyfluorinated arenes of interest, a number of common motifs were explored (FIG. 5). It was pleasingly found that electronically diverse range of aniline substituted tetrafluoropyridines underwent smooth HDF (5a′-5e′). However, aliphatic amino pyridine 5f′ were unreactive, possibly stemming from decreased acidity or inappropriate conformation due to the aliphatic nature of the substituent. Tetrafluoropyridine could be substituted with other important heterocycles such as pyridine to give partially fluorinated bipyridine, 5g′, after HDF. Additionally, a BOC-protected amine (5h) serves as an excellent directed group. Given the frequency of these motifs in pharmaceuticals and agrochemicals, the directed photo-HDF reaction may be useful for late stage defluorinations, and rapid substrate diversification in structure-activity relationship (SAR) studies.

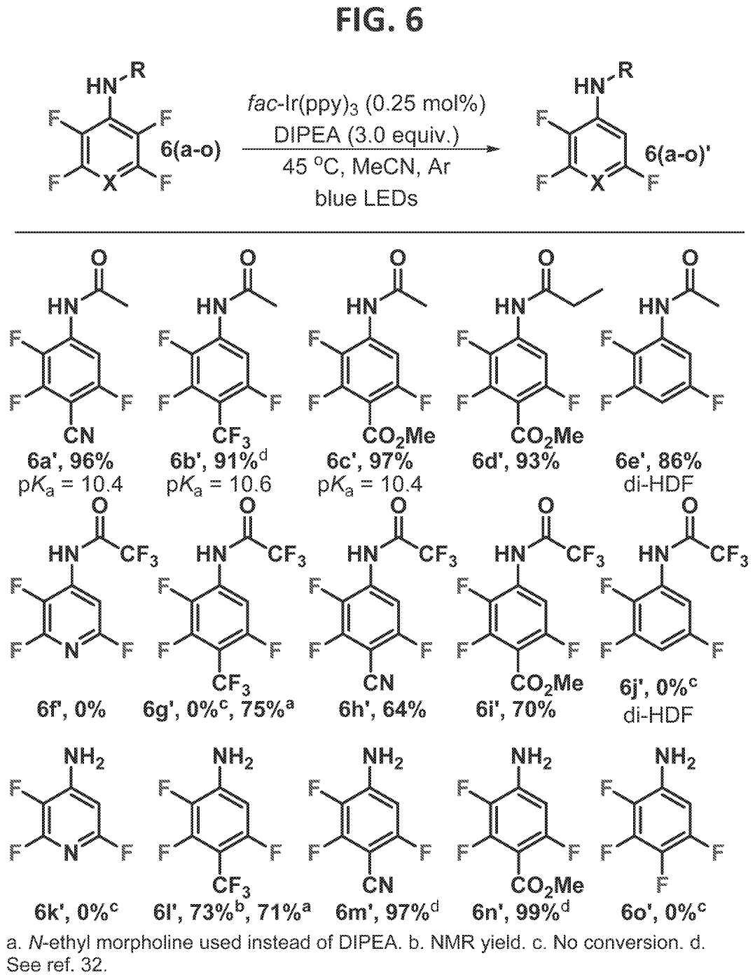

Next, the generality of the directed photo-HDF with respect to the polyfluoroarene was investigated (FIG. 6). It was pleasingly found that acetamide substituent could effectively direct the HDF on perfluorinated derivatives to give a single regioisomer, including benzonitrile (6a′), trifluorotoluene (6b′), benzoate (6c′), and even benzene (6e′). In the case of pentafluorobenzene derivative 6e, the initial mono-HDF was the directed product, but was rapidly consumed to give the di-HDF product (6e′).

Next, trifluoroacetyl was investigated as a directing group (6f-6j), which is attractive both because of its potential to acidify the NH, as well as its facile removal under basic conditions. While it worked well for several substrates, some substrates failed to form any product (6f, 6g, and 6j). In the case of 6f and 6g, the N—H was sufficiently acidified to be deprotonated by DIPEA, which retarded the rate of reaction (6f is nearly 4 pKa units more acidic than 4a; see Example 2 for further details). The consequence of N—H deprotonation would be a more difficult electron transfer to the perfluoroarene, since the original LUMO (lowest energy unoccupied orbital) would be occupied by the electrons originally located in the N—H bond, preempting electron transfer and fluoride fragmentation. Furthermore, the amine reductant would be protonated, preventing it from serving as the reductant. This possibility was probed by use of N-ethyl morpholine as the amine reductant, which is estimated to be nearly 3-4 pKa units less basic (MeCN) than DIPEA, as the reductant (Sobkowski et al., 2010). While the photo-HDF reaction of 6f still failed, the reaction of 6g took place smoothly, giving 6g′ in 75% yield. This result indicates that deprotonation of the directing group is one resolvable issue that can arise in the photo-HDF reaction. The reason for the failure of 6j is not clear at this time.

Substrates 6l and 6m show that even a simple amino group can often serve to facilitate the HDF event, provided there is an acidifying group attached to the ring (6l′-6n′). However, in the case of 4-amino pyridine (6k) and perfluoroaniline (6o), the reaction failed. Currently, the reasons for the failure of these substrates are abstruse.

The working hypothesis of the reaction is that the lifetime of the radical anion is relatively short, and productive fragmentation must compete with unproductive back electron transfer. Therefore, in order for the hydrogen bond to perturb the regiochemical outcome of the photo-HDF reaction, it must either be operative at the time of radical anion formation, or commence rapidly after the electron transfer event. Hence, the five-membered cyclic intramolecular hydrogen bond between the acidic proton and the neighboring fluoride would be ideal for achieving the directed HDF. Whether more remote hydrogen bond donors could effectively accelerate the fluoride fragmentation was yet to be seen. Consequently, this was systematically explored by inserting one or more atoms between the acidic N—H and the arene ring, enlarging the cyclic transition state from five to eight members.

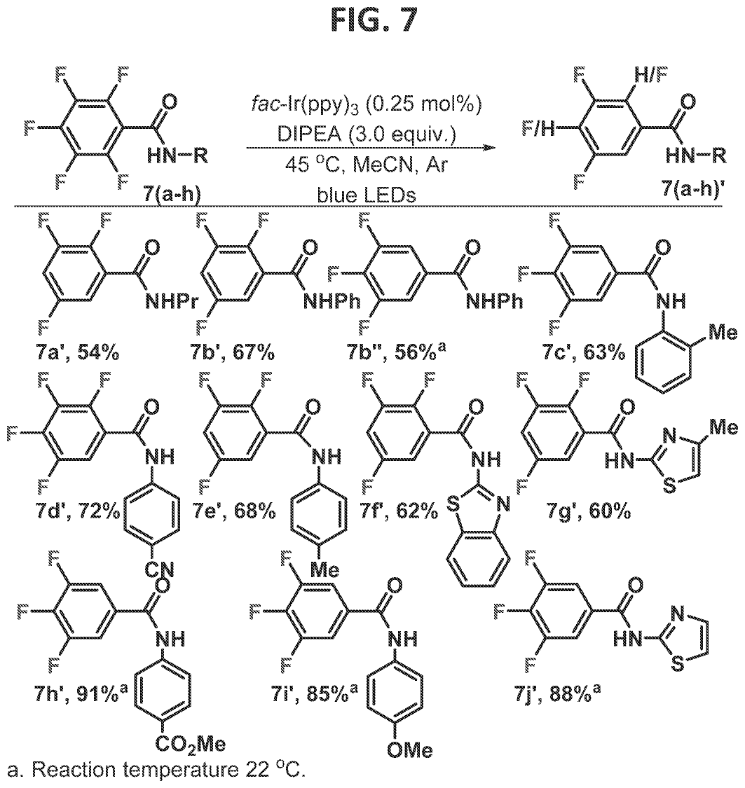

Next, this Example was expanded to include substrates which would form a six-membered intramolecular hydrogen bonding ring (FIG. 7). Specifically, perfluorobenzoylamides that can be synthesized in 1-step via amine addition to the perfluorobenzoyl chloride were subjected to the HDF conditions (7a-h). The perfluorobenzoyl amides, 7, were found to be more complex than other systems, in which several different reaction pathways could be operative, and conformational changes after one HDF event often facilitated the next HDF event. Careful monitoring of the reaction by ¹⁹F NMR revealed that simple aliphatic benzoyl amides (7a) first give the electronically controlled para-HDF product, which is rapidly consumed to give the di-HDF product in reasonable yield (7a′). Curiously, aniline derived amide 7b underwent directed HDF, and gave a temperature dependent product. At 45° C., the ortho/para di-HDF product was obtained (7b′), whereas when the reaction temperature was reduced to room temperature, the ortho/ortho′ di-HDF product was obtained (7b″).

When the more sterically congested amide 7c was subjected to the reaction, the ortho/ortho′ di-HDF product 7c′ was also obtained, even at 45° C. Increasing the acidity of the N—H further accelerated the directed HDF and allowed the isolation of the mono-HDF product 7d′ product in good yield (7d vs. 7e). Heteroaryl amides also served as good directing groups (7f′ and 7g′). Substrates 7h-7j highlight the important role that temperature plays in dictating regiochemistry. Specifically, at the slightly reduced temperature (i.e. 23° C.), high yields of the directed di-HDF product were obtained.

In several cases, directed mono-HDF was rapidly followed by a secondary electronically controlled HDF, i.e. para to the carbonyl group (7b, 7e-7g). Still in other substrates, the product outcome had a clear temperature dependence, and the electronically controlled HDF could be avoided all together (7b-7d, and 7h-7j). It was suspected that these divergent reaction outcomes were primarily due to conformational changes associated with the orientation of the directing group which occur after the first HDF event, and were precipitated by decreased steric repulsion about the carbonyl group upon substitution of the fluorine with hydrogen. The increased flexibility, in turn, could allow increased conjugation between the n-system of the carbonyl and fluoroarenes, which would lead to a lower lying LUMO, and therefore a more facile electron transfer to the arene. However, it would place the N—H in the plane of the fluoroarene ring, or nearly in the plane of the fluoroarene ring, preventing the key C—F deformation needed to accomplish directed-HDF. Consequently, electronically controlled fragmentation would dominate. It was postulated that it might be possible to find an amide with a structure such that even after the first HDF event, it would not be prone to undergo the hypothesized conformationally dictated electronic HDF. Accomplishing this could provide an unprecedented level of control. Such control is desperately needed if C—F functionalization is to become reality in the synthesis of multifluorinated arenes.

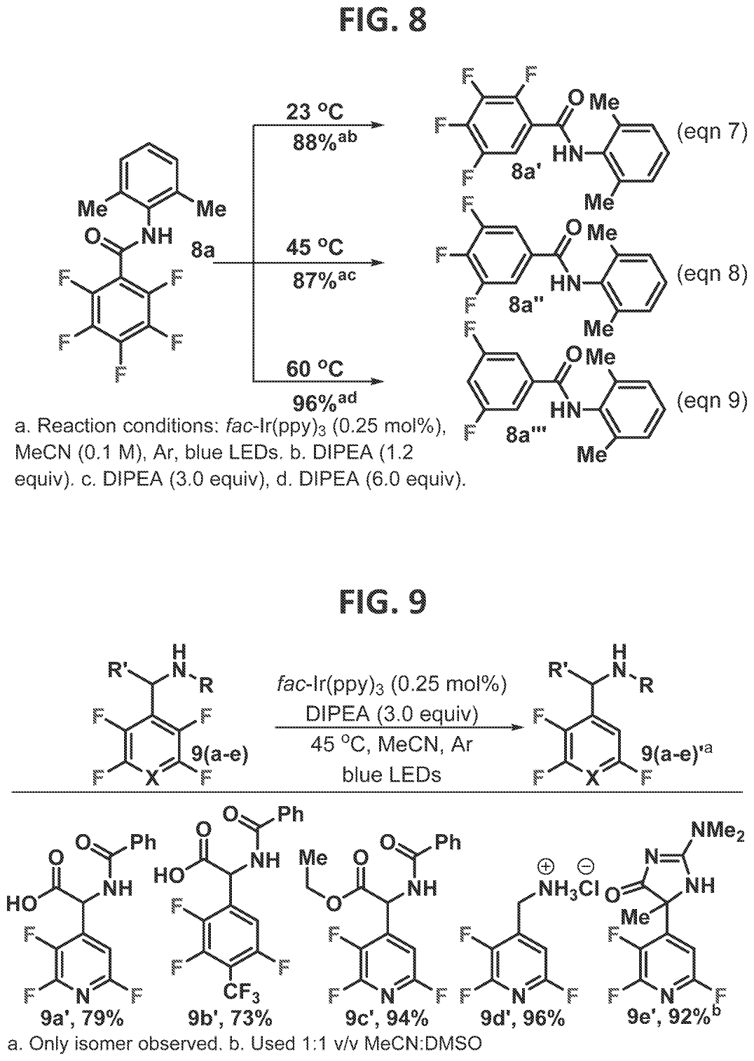

Lloyd-Jones and Booker-Milburn have shown that very bulky amides are prone to undergo solvolysis. The reason for this is facile N—CO bond rotation which results from steric decompression which occurs as the N rotates out of conjugation with the amide (Hutchby et al., 2012). While the system of the present disclosure is somewhat different, it was hoped that a bulky amide could be used to more easily achieve the necessary out of plane N—H, which was posited would lead to directed-HDF. Thus, sterically hindered amide 8a (FIG. 8) was synthesized, which indeed provided excellent thermal control over the product distribution. At room temperature, the reaction gave primarily ortho-HDF (eqn 7), along with a small amount of 8″. Simply heating gently (45° C.) and extending the reaction time, the same substrate could be enticed to undergo di-HDF to obtain the 2,6-di-HDF product, 8a″, in 87% (eqn 8). Finally, at 60° C. with 6.0 equivalents of DIPEA, it was observed that the ortho/para/ortho′ tri-HDF product could be obtained (eqn 9). Thus, the 2,6-dimethyl aniline derived amide (8a) is a very versatile substrate that can provide facile access to several fluorinated derivatives simply by modulating reaction temperature, time, and amine loading as the key reaction parameters.

While not every possible fluorination pattern of pentafluorobenzoic acid has yet been realized, collectively, these results highlight the versatility of the photocatalytic C—F reduction strategy. By judicious choice of (non)directing group, in just two synthetic steps from commercially available perfluorobenzoyl chloride, five different polyfluorination patterns can be obtained in high yield, and is a realization of significant progress in the efforts to synthesize multifluorinated arenes.

The study of substrates which involved 6-membered hydrogen bonds was expanded by exploring non-natural alpha amino acids derivatives (FIG. 9), which are rapidly synthesized via perfluoroarylation of the oxazolone followed by the appropriate workup (Teegardin and Weaver, 2017), or in the case of 9d via the addition of nitromethane followed by hydrogenation (Day and Weaver, 2017). It was pleasingly found that the benzoyl protected amino acids (9a and 9b) underwent smooth photo-HDF, and that the HDF products were isolated in good yields. The ethyl ester derivative was isolated in high yield (9c′). Somewhat surprisingly, the HCl salt of the benzylic amine (9d) proved to be an excellent substrate, even if the reaction was somewhat sluggish due to its low solubility. Protonation of the amine was found to be essential for reactivity. Given the propensity for primary amines to undergo nucleophilic aromatic substitution with these types of arenes, it is particularly remarkable that not only did the protonation of the amine (HCl salt) serve as a protecting group, but also as an excellent directing group (9d′).

It is conceivable that the ammonium salt accelerates the electron transfer via electrostatic stabilization of the radical anion, in addition to the fluoride fragmentation. In contrast to previous examples, fluoride fragmentation would yield an overall neutral complex upon complete protonation of the leaving fluoride. It was pleasingly found that the perfluoroaryl cyclic guanidine (9e), which is a motif that is under investigation as a potential therapeutic for Alzheimer's disease as a BACE-inhibitor (Meyers et al., 2014), undergoes smooth directed-HDF to the product 9e′ in high yield. The ability to rapidly vary the fluorination pattern will be helpful in lead optimization during discovery efforts involving fluorinated arenes.

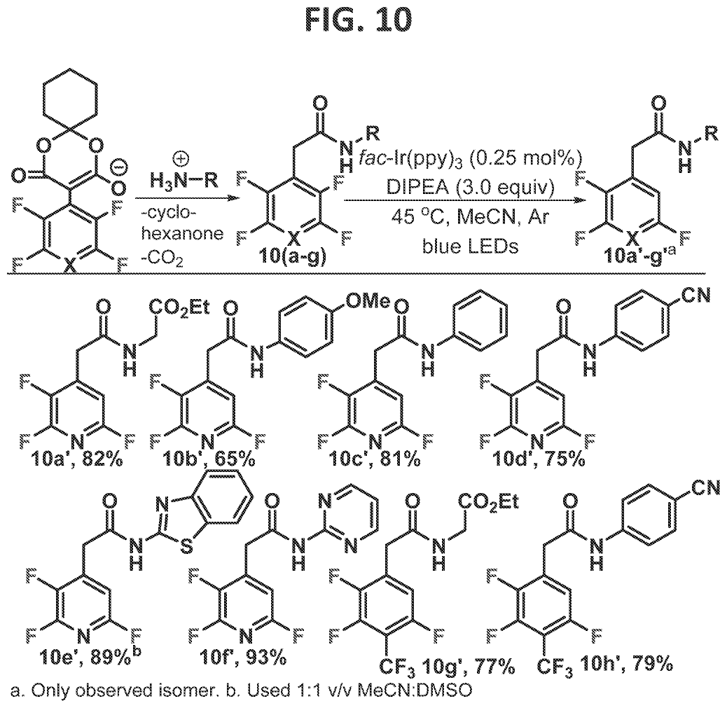

Next, systems in which a seven membered hydrogen bond ring is formed were investigated (FIG. 10). The substrates are formed in a single step (Senaweera et al., 2014) via the decomposition of the perfluoroarylated Meldrum's acid enolate salts, which are commercially available as FAYE blocks (Fluoroaryl AcYI Equivalents; Cat No. 300852 through Aspira Scientific (Milpitas, Calif.), and Cat No. 809268 through Sigma-Aldrich (St. Louis, Mo.)). As seen in smaller ring systems involving the tetrafluoropyridine motif (i.e. 4a, 5a, and 9a), only a single regioisomer was observed in the directed-HDF reaction across a diverse range of directing groups, giving the trifluoropyridine in good isolated yields (10a′-10r, 10d′). Additionally, the glycine ethyl ester (10g) and benzonitrile aniline (10h) derived heptafluorotoluenes underwent smooth directed-HDF to give hexafluorotoluene products (10g′ and 10h′) in good yield and perfect selectivity.

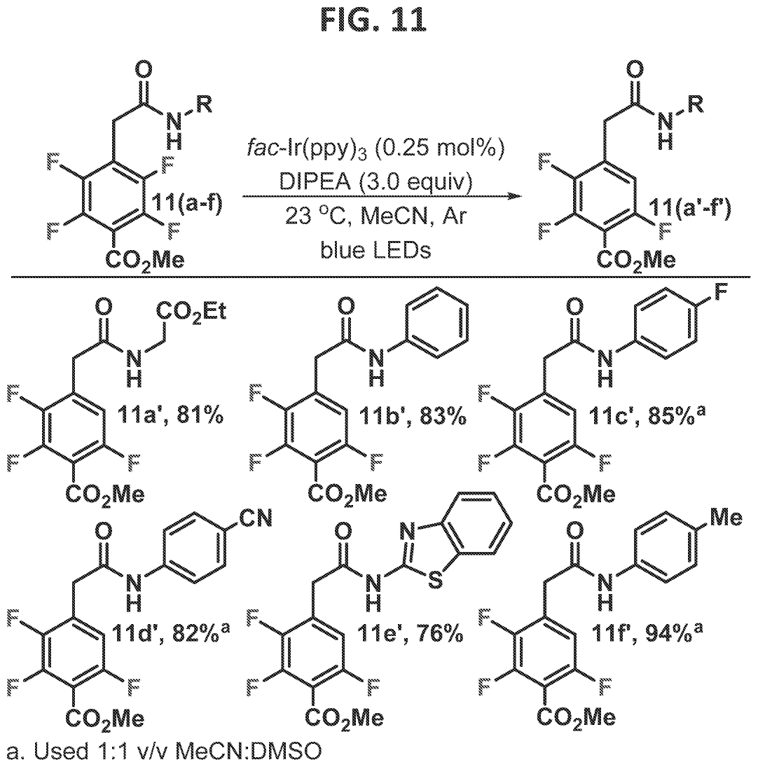

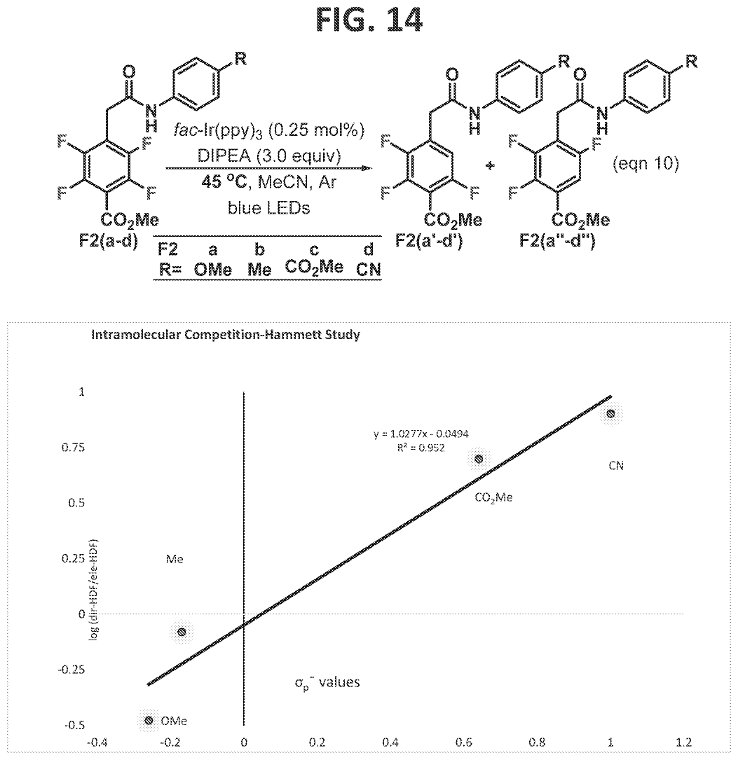

Next, the benzoate series was investigated (FIGS. 11 and 14). In the inventor's experience, methyl pentafluorobenzoate is more prone to a second electronically controlled photocatalytic fragmentation event when compared to other perfluoroarenes (Senaweera et al., 2014a; Senaweera et al., 2014b). The second fragmentation takes place at the position ortho to the carboxy group. It is not surprising, then, that when the preferred site of photocatalytic functionalization is substituted with a carbon substituent para to the ester, the electronically controlled HDF still takes place at a reasonable rate. Thus, substitution of the para position of the benzoate system with a directing group presented an opportunity for an internal competition experiment. This was investigated by conducting an experiment to compare the relative rate between the directed- and electronically-controlled HDF and is reflected in the regioselectivity (FIG. 14, eqn 10). When the log of the ratio of the directed-HDF product (F2a′-d′) to the electronically controlled-HDF product (F2a″-d″), obtained at 45° C., was compared to the Hammett values, a linear correlation with a positive slope (p=1.027) was observed. This finding is consistent with the earlier Hammett study within the five membered series (FIG. 13), which also indicated negative charge build up in the transition state. When R is an electron releasing group, the electronically controlled product (F2a″-d″) is dominant. In contrast, electron withdrawing substituents such as a nitrile accelerate the directed-HDF reaction (F2a′-d′). A positive p value suggests that negative charge increases in the transition state and explains why the acidity of the proton plays an important role in determining which fluorine undergoes fragmentation.

Next, a range of directing groups were explored within the methyl benzoate series in which a seven membered hydrogen bond is formed (FIG. 11). Again, a significant temperature effect was observed, similar to what was previously observed for the perfluorobenzoyl substrates (FIGS. 7 and 8). For instance, when 11f was used in the Hammett study at 45° C., the electronically controlled product was favored (F2b″ 1.2:1, FIG. 14). However, by dropping the temperature to 23° C., the directed-HDF product was formed almost exclusively (94%, 11f′, FIG. 11). Thus, the photo-HDF reaction of all of the benzoate substrates was performed at room temperature, in order to favor the directed-HDF product.

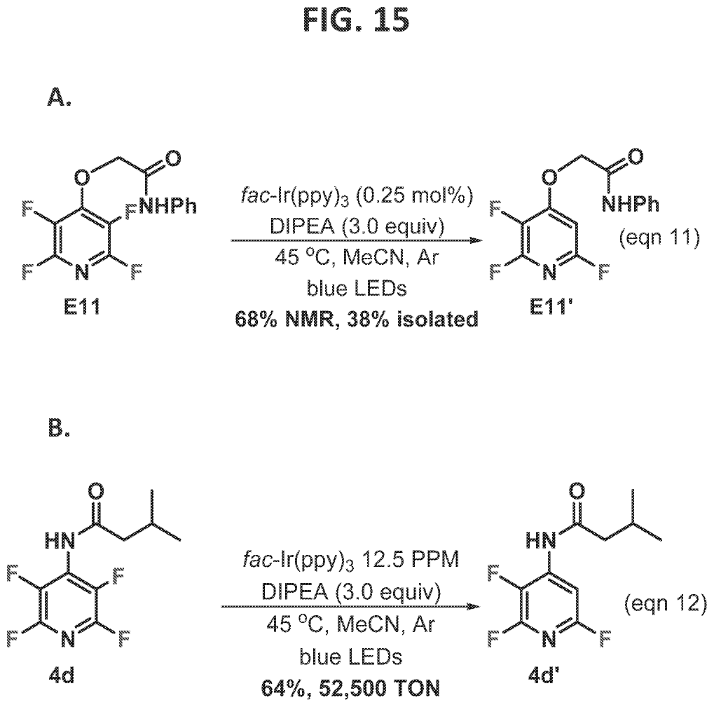

Finally, the ability to perform the directed photo-HDF when the directing group formed an 8-membered hydrogen bonding cycle was briefly evaluated (FIG. 15A, eqn 11). While the reaction was noticeably slower, and did not reach completion, it did give the directed-HDF product exclusively, demonstrating the ability to use even relatively remote acidic groups to facilitate the reaction. This motif is found in a number of aryloxyacetic acid drugs, such as ethacrynic acid and fenofibrate, and represents a major class herbicides such as 2,4-D, and fluoroxypyr. Thus, the directed photo-HDF reaction may be helpful in accelerating compound discovery that encompasses future elaboration of this motif toward fluorinated congeners.

The reaction may also prove useful for larger scale production. Two obvious issues are the batch nature of the reaction and the scarcity of rare earth metals, iridium in this case, that are used in the photocatalyst. First, the inventor (Senaweera et al., 2014a) and countless others (McTeague et al., 2016; Straathof et al., 2016; Staveness et al., 2016; Rackl et al., 2016; Hernandez-Perez et al., 2016; and Cantillo et al., 2014) have demonstrated that almost any photo-HDF reaction can be transposed from batch to flow methodology, usually with improved kinetics. Second, given that the reaction is triggered by an electron transfer, it is likely that there are numerous other catalysts which could facilitate the photo-HDF reaction and relieve the iridium issue altogether (Lu et al., 2016). Until that is demonstrated, it was desired to probe the robustness of the photocatalytic system for this reaction (FIG. 15B, eqn 12). In the standard reaction conditions, relatively low catalyst loadings (0.25 mol %) were used, although presumably much lower Ir-loading would be needed in order for the process to be amenable to scaling. Remarkably, with reduced photocatalyst loading but otherwise normal reaction conditions (12.5 ppm or 0.00125 mol % Ir(ppy)₃), 64% assay yield was observed, giving 52,500 catalytic turnovers (TONS), demonstrating that it may be possible to use even an Ir-based photocatalyst to accomplish a commercially important hydrodefluorination.

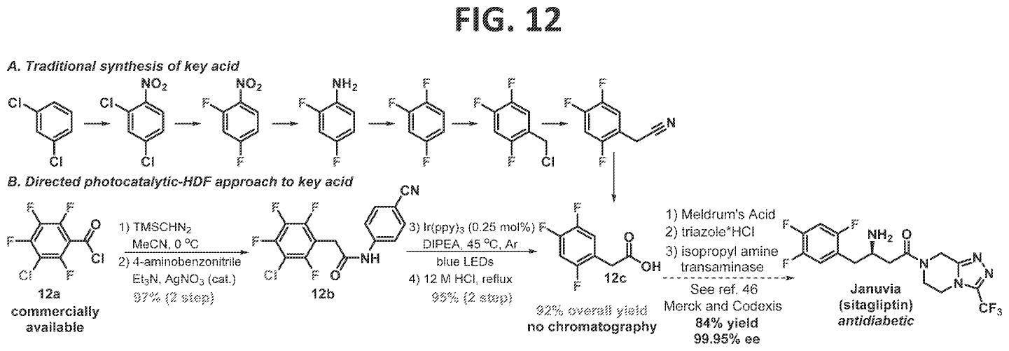

Having developed a solid understanding of the directed and undirected photo-HDF reaction, it was desired to demonstrate the potential of the reaction, and more generally the potential of the C—F functionalization/reduction strategy to facilitate access to important multifluorinated arenes. As a target, it was chosen to synthesize the key starting trifluorophenyl acetic acid (12c, FIG. 12) which was utilized by Merck and Codexis in the third generation synthesis of the anti-diabetic drug, JANUVIA® (sitagliptin; Savile et al., 2010). While the end-game of the sitagliptin synthesis is truly elegant, the synthesis of the key acid 12c is wanting, and thus, represents an ideal platform to demonstrate the utility of the methodology of the present disclosure.

This synthesis began with 3-chloro-tetrafluorobenzoyl chloride (12a, FIG. 12), which contains all of the necessary fluorines in the desired positions and is likely derived in just a few steps from benzonitrile (the 3-chloro-tetrafluorobenzoyl chloride currently costs $11.60/gram from Alfa Aesar (as of Apr. 14, 2017)). Three main objectives needed to be accomplished: homologation of the acid, photocatalytic dechlorination, and directed photocatalytic defluorination. The homologation step can come before or after the dehalogenations, as both lead to formation of the product. It was elected to first convert the benzoic acid derivative to an acetic acid derivative by forming the α-diazo ketone, which was subjected without isolation to a silver catalyzed, one-step Wolff rearrangement-amidation sequence to arrive at 12b in 97% crude yield. It was now positioned to perform the key photocatalytic di-dehalogenation (HDX) reaction, in which removal of the chlorine at the 3-position and the fluorine at the 6-position were needed. Without any chromatographic purification, the material was taken into the photocatalytic-HDX reaction. Based on the inventor's experience, it was expected that the chlorine would fragment first (Senaweera et al., 2014; Singh et al., 2015; Senaweera et al., 2016; and Singh et al., 2014), which would lead to an unsymmetric intermediate. Fluorines at both the 2 and the 6 positions could undergo HDF. It was hoped that the reduction of the chlorine would electronically activate the fluorine at the 6-position, as this had been previously seen in the benzene series (Senaweera et al., 2014; and Lu et al., 2016). After full conversion, the solvent was removed and the crude material refluxed in aqueous HCl. Purification via acid/base workup removed the photocatalyst, and the aniline to afford the analytically pure product 12c in 95% yield, and 92% overall yield from a commercially available benzoyl chloride.

The current telescoped synthesis required no column chromatography and gave an overall yield of 92% yield from 12a, and is the most expedient synthesis to this important trifluorinated acid. To the inventor's knowledge, this is the first use of a defluorination strategy to access a commercially interesting multifluorinated arene, and will allow for incorporation of this defluorination as a strategy to access important multifluorinated compounds.

Conclusions of Example 1

This Example has demonstrated and explored the ability of hydrogen bonding to accelerate and alter the regioselectivity of the photocatalytic-HDF reaction. This work provides access to complimentary regioselectivity compared to the previously disclosed electronically dictated outcome, which is key for furthering the synthetic strategy of C—F functionalization. Though the reaction takes place through a photocatalyzed outer sphere electron transfer, which to some extent may limit the ability to control the reaction outcome, it has been shown that careful planning, and utilization of the commonly encountered acidic N—H, can allow control over the reaction outcome, even when many isomers are possible. Furthermore, this strategy may be applied to other radical anion fragmentation chemistry to help overcome the normal selectivities.

### Example 2—Supplemental Information for Example 1

General Experimental

All reagents were obtained from commercial suppliers (Sigma-Aldrich, Oakwood chemicals, Alfa Aesar, and Matrix Scientific) and used without further purification unless otherwise noted. Acetonitrile (CH₃CN) was dried over molecular sieves. Diisopropylethylamine was purchased from Sigma Aldrich. Photocatalyst tris(2-phenyl pyridinato-C²)iridium(III) (Ir(ppy)₃) was synthesized according to literature procedure (Singh et al., 2015). Reactions were monitored by ¹⁹F NMR and GC-MS (QP 2010S, Shimadzu equipped with auto sampler). NMR spectra were obtained on a 400 MHz Bruker Avance III spectrometer or a 400 MHz Unity Inova spectrometer. ¹H and ¹⁹F NMR chemical shifts are reported in ppm relative to the residual solvent peak. Purifications were carried out using Teledyne Isco Combiflash Rf 200i flash chromatograph with REDISEP® Rf normal phase silica (4 g, or 12 g; Teledyne ISCO, Lincoln, Nebr.). Substrate synthesis reactions were monitored by thin layer chromatography (TLC), obtained from Sorbent Technology Silica XHL TLC Plates, w/UV254, glass backed, 250 μm, and were visualized with ultraviolet light.

Photocatalytic Reaction Set Up

Photocatalytic reactions were set up in a light bath as described below. Strips of blue LEDs (18 LED/ft.) were purchased from Solid Apollo. The strips (4.9 ft) were wrapped around on the walls of a glass crystallization dish and secured with masking tape and then wrapped with aluminum foil. A lid which rests on the top was fashioned from cardboard, and holes were made such that NMR tubes were held firmly in the cardboard lid, which was placed on the top of bath. Water was added to the bath such that the tubes were submerged in the water which was maintained at the appropriate temperature.

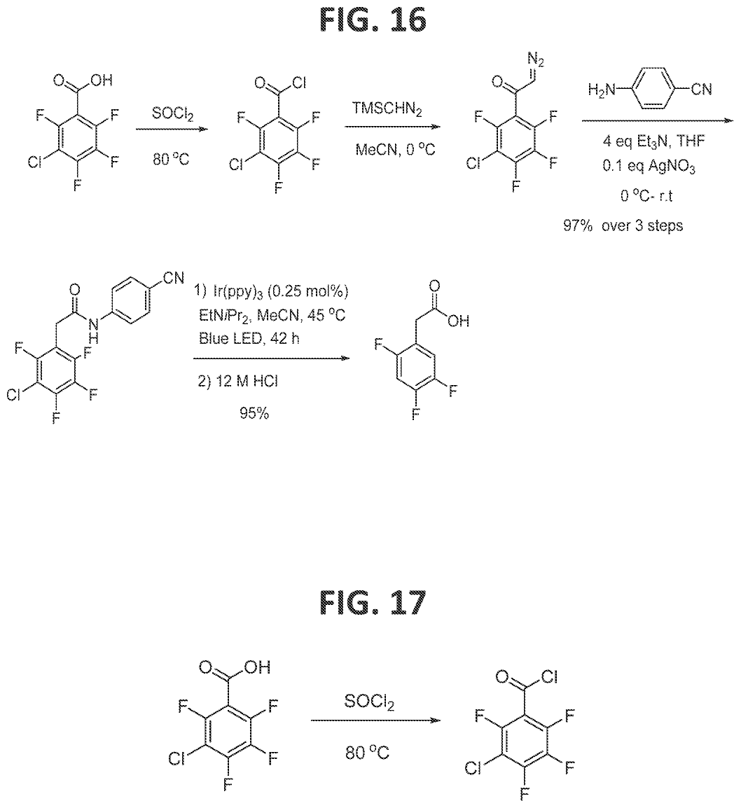

Second generation synthesis of 2,4,5-trifluorophenylacetic acid (FIG. 16) and synthesis of 3-chloro-2,4,5,6-tetrafluorobenzoyl chloride (FIG. 17): Under an atmosphere of argon and anhydrous conditions, excess thionyl chloride (2 mL) was added to commercially available 3-chloro-2,4,5,6-tetrafluorobenzoic acid (300 mg, 1.32 mmol) in a 25 mL round-bottomed flask equipped with a reflux condenser. The mixture was gently stirred and refluxed in an oil bath for 2 h. After cooling the reaction mixture to room temperature, excess thionyl chloride was evaporated under vacuum (780 mmHg), leaving corresponding acid chloride in the flask as a pale yellow residue. The product is moderately volatile, and care should be taken not to evaporate it. The product was taken to the next step without any further purification.

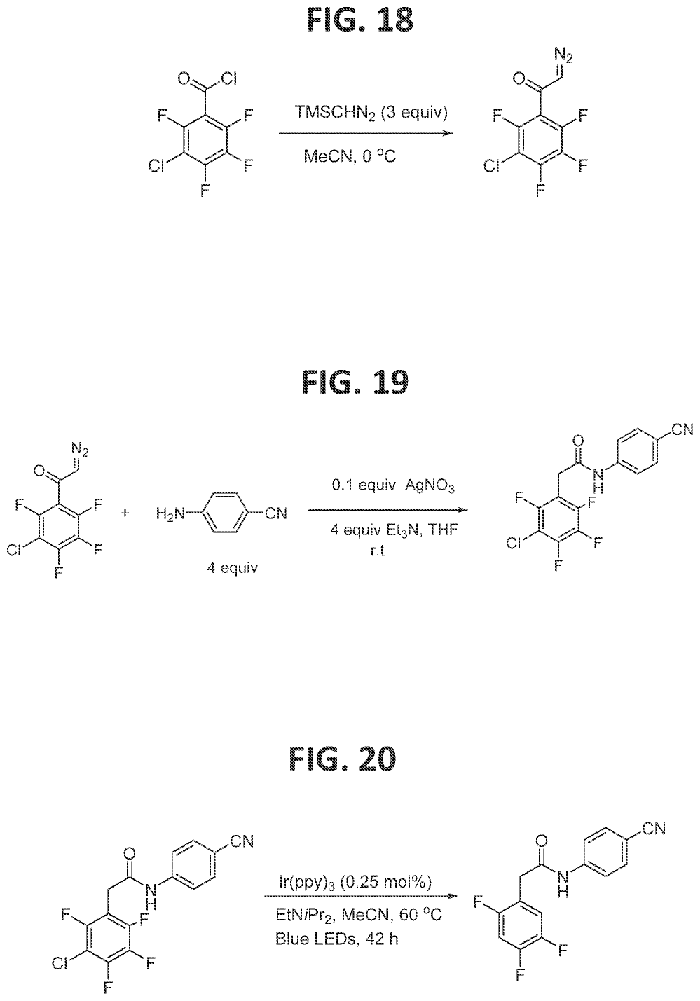

Synthesis of 1-(3-chloro-2,4,5,6-tetrafluorophenyl)-2-diazoethan-1-one)(FIG. 18): Under an atmosphere of argon and anhydrous conditions, 3-chloro-2,4,5,6-tetrafluorobenzoyl chloride was dissolved in MeCN (5 mL) in a dry 25 mL round bottom flask. Trimethylsilyl diazomethane (2 M solution in diethyl ether, 2 mL, 3.9 mmoles, 3 equiv.) was added dropwise via syringe to the reaction mixture at 0° C. over 15 minutes. After stirring at 0° C. for 30 minutes, the yellow solution was stirred at ambient temperature for 7 h. The MeCN and volatiles were then removed under reduced pressure (780 mmHg), resulting in a residue. The product, 1-(3-chloro-2,4,5,6-tetrafluorophenyl)-2-diazoethan-1-one, was used in the next step of the sequence without further purification.

Synthesis of 2-(3-chloro-2,4,5,6-tetrafluorophenyl)-N-4-cyanophenyl)acetamide (FIG. 19): Under an atmosphere of argon and anhydrous conditions, in a 25 mL round bottom flask equipped with a magnetic stir bar, and previously synthesized 1-(3-chloro-2,4,5,6-tetrafluorophenyl)-2-diazoethan-1-one (320 mg, 1.27 mmol) was added THF (10 mL) and 4-aminobenzonitrile (600 mg, 5 mmol, 4 equiv), then triethylamine (0.7 mL, 5 mmol, 4 equiv) was added slowly, at 0° C., with constant stirring, after which all of the solid material was dissolved. Silver nitrate (22 mg, 0.13 mmol, 0.1 equiv) was added into the reaction mixture. Aluminum foil was used to cover the reaction flask. The reaction mixture was sonicated for 1.5 hours, and then the mixture was stirred at ambient temperature for another 2.4 h. The reaction was quenched by the addition of 1M HCl (8 mL). The mixture was extracted with Et₂O (3×20 mL), and the combined organic layers were washed with brine, dried over MgSO₄, filtered, and evaporated to dryness under reduced pressure, to afford the 2-(3-chloro-2,4,5,6-tetrafluorophenyl)-N-(4-cyanophenyl)acetamide as a yellow powder in 97% yield (421 mg, 1.2 mmol).

Synthesis of N-(4-cyanophenyl)-2-(2,4,5-trifluorophenyl)acetamide (FIG. 20): An NMR tube capped with NMR septa (Ace glass, part no. 9096-25) was charged with (Ir(ppy)₃) (0.25 mM, 1 mL in MeCN), 2-(3-chloro-2,4,5,6-tetrafluorophenyl)-N-(4-cyanophenyl)acetamide (34 mg, 0.1 mmol, 1 equiv), and N,N-diisopropylethylamine (2 equiv), and the tube was capped and the reaction was degassed at 0° C. to avoid evaporation of N,N-diisopropylethylamine during the degassing process (Ar bubbling for 10 min). The NMR tube was placed in a light bath (vide supra), and the lower portion of the tube was submerged under the water bath which was maintained at 60° C. The reaction was monitored by ¹⁹F NMR. After the complete conversion of the starting material to product, the CH₃CN was removed via rotary evaporation (rotovap), and the residue was used in the subsequent reaction without further purification.

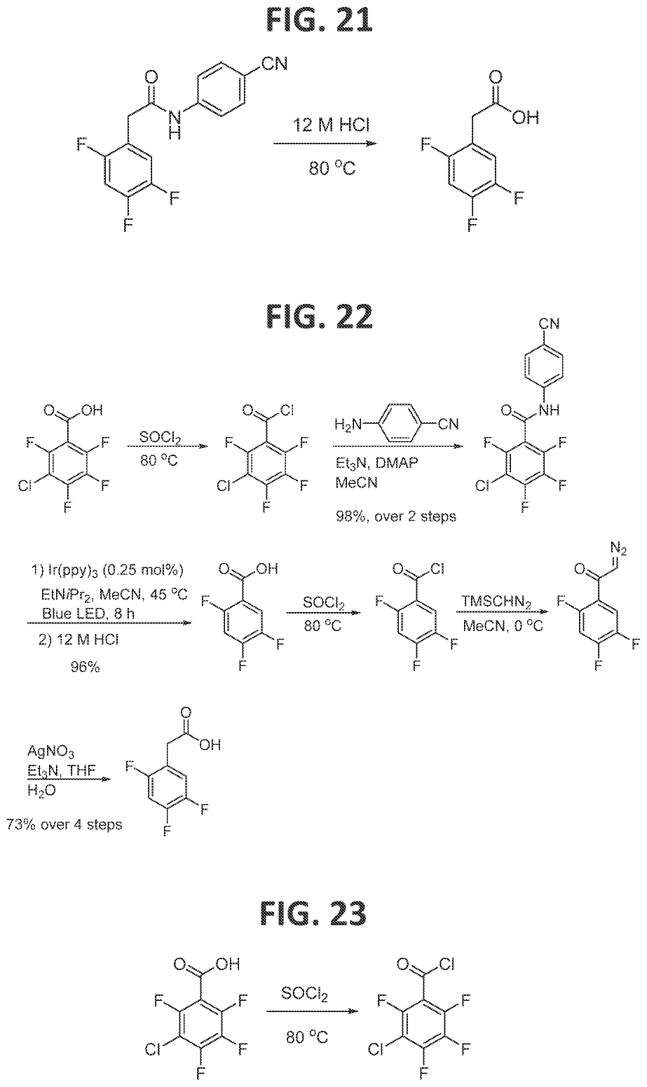

Synthesis of 2,4,5-trifluorophenylacetic acid (FIG. 21): In an NMR tube capped with NMR septa (Ace glass, part no. 9096-25) was charged with N-(4-cyanophenyl)-2-(2,4,5-trifluorophenyl) acetamide and 12 M HCl. The NMR tube was placed in an oil bath and heated to 80° C. for 5 h. The reaction mixture was cooled to room temperature, and then the aqueous phase was extracted with dichloromethane (3×8 mL), and the combined organic layers were then treated with 1 M NaOH (3×8 mL). The combined aqueous layers residue were acidified with 2 M HCl and then extracted with chloroform (3×10 mL). The combined layers were dried with anhydrous magnesium sulfate, and concentrated under reduced pressure to furnish a white powder in 95% yield (18 mg, 0.09 mmol).

Scheme of first generation synthesis of 2,4,5-trifluorophenylacetic acid (FIG. 22) and synthesis of 3-chloro-2,4,5,6-tetrafluorobenzoyl chloride (FIG. 23): The compound was prepared as described above.

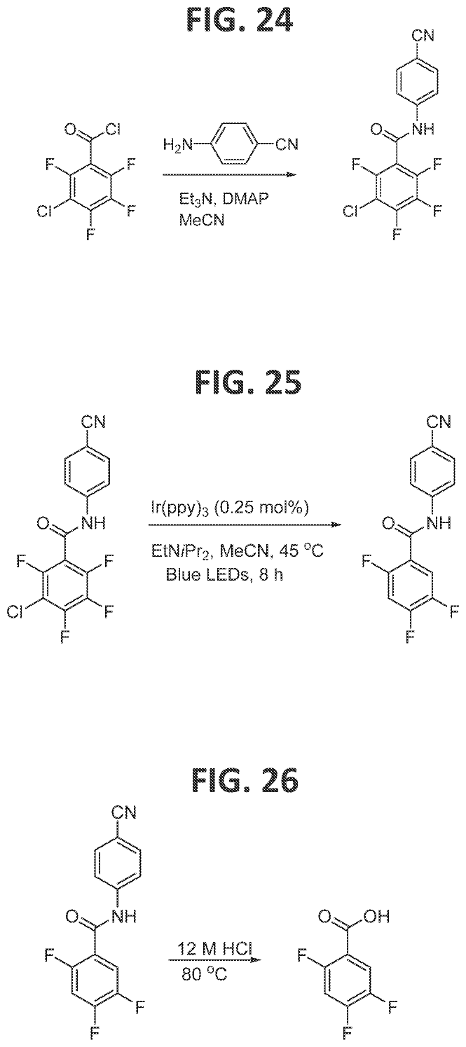

Synthesis of 3-chloro-N-(4-cyanophenyl)-2,4,5,6-tetrafluorobenzamide (FIG. 24): Under an atmosphere of argon and anhydrous conditions, an ice-cooled 25 mL round-bottomed flask containing a solution of 4-aminobenzonitrile (576 mg, 4.87 mmol, 1.20 equiv) and a catalytic amount of DMAP (20 mg) in dry THF (20 mL) were added. Then triethylamine (680 μL, 4.87 mmol, 1.20 equiv) and the 3-chloro-N-(4-cyanophenyl)-2,4,5,6-tetrafluorobenzamide (4.80 mmol, 1.0 equiv) were added. After 10 min, the ice bath was removed, and the reacting mixture was allowed to warm to room temperature (10 h). After this time, the resulting mixture was poured into saturated NH₄Cl solution (20 mL). The layers were separated and the amide extracted from the aqueous layer Et₂O (3×20 mL). The combined organic phases were washed with 1 M HCl (12 mL), brine (20 mL), dried over MgSO₄ (4 g), filtered, and evaporated to dryness under reduced pressure to furnish the product as a yellow powder in 98% yield (1.30 g, 3.7 mmol).

Synthesis of 2,4,5-trifluorobenzoic acid (FIG. 25): In an NMR tube capped with NMR septa (Ace glass, part no. 9096-25) was charged with (Ir(ppy)₃) (0.25 mM, 1 mL in MeCN), 3-chloro-N-(4-cyanophenyl)-2,4,5,6-tetrafluorobenzamide (33 mg, 0.1 mmol, 1 equiv), and N,N-diisopropylethylamine (2 equiv), and the reaction was degassed at 0° C. to avoid evaporation of N,N-diisopropylethylamine during the degassing process (Ar bubbling for 10 min). The NMR tube was placed in a light bath (vide supra), and the lower portion of the tube was submerged under the water bath, which was maintained at 45° C. The reaction was monitored by ¹⁹F NMR, and after the complete consumption of starting material, the CH₃CN was removed via rotovap, and the residue was used in the following steps without further purification.

Synthesis of 2,4,5-trifluorobenzoic acid (FIG. 26): In an NMR tube capped with NMR septa (Ace glass, part no. 9096-25) was charged with N-(4-cyanophenyl)-2,4,5-trifluorobenzamide and 12 M HCl. The NMR tube was placed in an oil bath and heated to 80° C. for 5 h. The reaction mixture was cooled to room temperature, and the aqueous phase was extracted with dichloromethane (3×8 mL), the combined organic layers were then treated with 1 M NaOH (3×8 mL). The combined aqueous layers residue were acidified with 2 M HCl, and the aqueous phase was extracted with chloroform (3×10 mL). The combined organic layers were dried with anhydrous magnesium sulfate and concentrated under reduced pressure to furnish the product as a pale yellow powder in 96% yield (17 mg, 0.10 mmol).

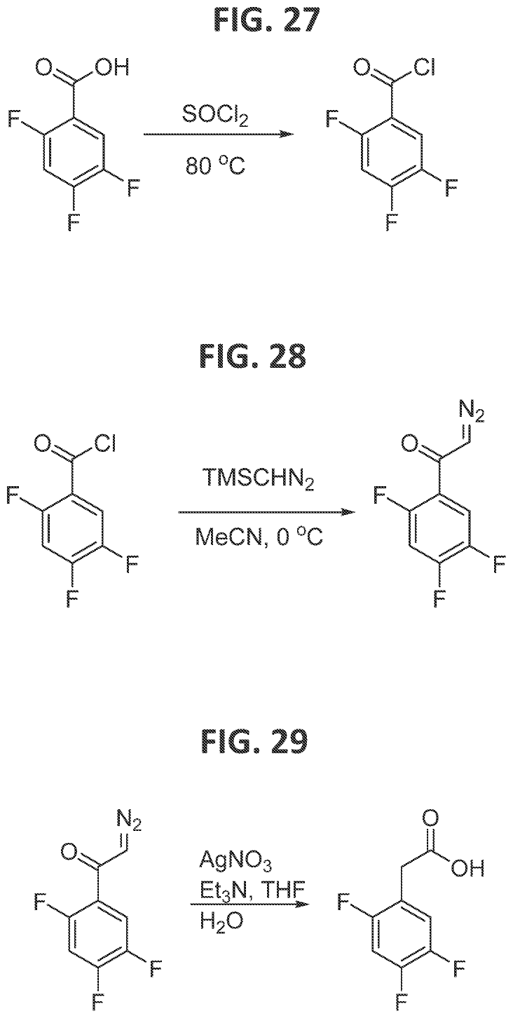

Synthesis of 2,4,5-trifluorobenzoyl chloride (FIG. 27): To 2,4,5-trifluorobenzoic acid (80 mg, 0.45 mmol) in a 5 mL round-bottomed flask and equipped with a reflux condenser was added excess thionyl chloride (2 mL). The mixture was gently stirred and refluxed in an oil bath for 2 h. After cooling the reaction mixture to room temperature, excess thionyl chloride was evaporated under vacuum (780 mmHg), leaving the benzoyl chloride behind as a pale yellow residue. The product was taken onto the next step without further purification.

Synthesis of 2-diazo-1-(2,4,5-trifluorophenyl)ethan-1-one (FIG. 28): Under an atmosphere of argon and anhydrous conditions, the 2,4,5-trifluorobenzoyl chloride (87 mg, 0.45 mmol) was dissolved in MeCN (5 mL) in a 25 mL round bottom flask. Trimethylsilyl diazomethane (2M solution in diethyl ether, 0.67 mL, 1.3 mmoles, 3 equiv) was added dropwise via syringe to reaction mixture at 0° C., over 15 minutes. After stirring at 0° C. for 30 minutes, the yellow solution was allowed to warm to ambient temperature and continued stirring (6 h). The MeCN and resulting residue were then removed under reduced pressure (780 mmHg). The 1-(3-chloro-2,4,5,6-tetrafluorophenyl)-2-diazoethan-1-one intermediate was used in the next step of the reaction without further purification.

Synthesis of 2-(2,4,5-trifluorophenyl)acetic acid (FIG. 29): Under an atmosphere of argon and anhydrous conditions, a 25 mL round bottom flask equipped with a magnetic stir bar and 2-diazo-1-(2,4,5-trifluorophenyl)ethan-1-one (89 mg, 1.27 mmol), was charged with THF (7 mL). Then triethylamine (0.25 mL, 1.8 mmol, 4 equiv) was added slowly at 0° C. with constant stirring, after which all of the solid material was dissolved. Silver nitrate powder (7.5 mg, 0.13 mmol, 0.1 equiv) was added into the reaction mixture. Aluminum foil was used to cover the reaction flask, and after the reaction mixture was sonicated for 1.5 h, H₂O (0.03 mL, 1.8 mmol, 4 equiv) was added dropwise over 10 minutes. Then, the mixture was stirred at ambient temperature for another 3 h before the reaction was quenched by addition of 1M HCl (8 mL). The mixture was extracted with Et₂O (3×20 mL), the combined organic layers washed with brine, and dried over MgSO₄. Filtration and concentration followed by purification by flash column chromatography (SiO₂, 20% ethyl acetate/hexanes) afforded the 2-(2,4,5-trifluorophenyl)acetic acid as a yellow powder in 89% yield (75 mg, 1.2 mmol).

### Example 3—Synthesis of Sitagliptin

JANUVIA® (sitagliptin, Merck & Co., Inc., Kenilworth, N.J.) is a popular diabetes drug that generates $6 billion in annual sales for its maker, Merck & Co., Inc. (Kenilworth, N.J.). The IUPAC name for sitagliptin is (3R)-3-amino-1-[3-(trifluoromethyl)-6,8-dihydro-5H-[1,2,4]triazolo[4,3-a]pyrazin-7-yl]-4-(2,4,5-trifluorophenyl)butan-1-one.

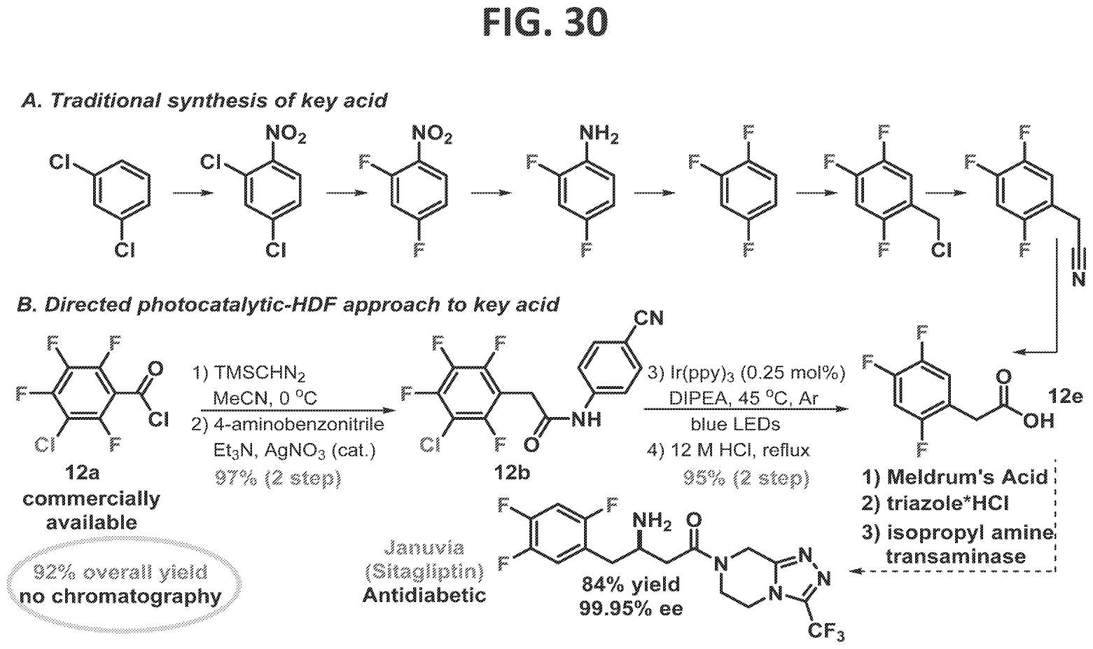

FIG. 30 compares the synthesis of the antidiabetic JANUVIA® (sitagliptin, Merck & Co., Inc., Kenilworth, N.J.) acid by the traditional prior art method (upper scheme A; Savile et al., 2010) and by the directed photocatalytic-HDF approach to key acid methods of the present disclosure (lower scheme B). As can be seen, the traditional synthesis requires seven steps to arrive at the key acid intermediate (12e). In contrast, the directed photocatalytic-HDF approach utilizes a commercially available, relatively inexpensive starting reagent and only involves two telescoped steps to arrive at the key acid intermediate. The synthesis began with 3-chloro-tetrafluorobenzoyl chloride (12a), which contains all of the necessary fluorines in the desired positions and is relatively inexpensive. First, a Wolff rearrangement was utilized to facilitate a homologation and install an appropriate directing group (12b), and next, the photocatalytic reduction removed both the chlorine and the undesired fluorine which was then hydrolyzed to yield analytically pure key acid (12e).

This directed photocatalytic-HDF approach provided a 92% overall yield without any chromatography steps involved.

### Example 4—Synthesis of Fluconazole

DIFLUCAN® (fluconazole, Pfizer Consumer Healthcare, Mississauga, ON) is a triazole antifungal drug that is used to treat a variety of fungal infections in humans and veterinary subjects; Fluconazole is the most widely available and commonly used agent against Candida infection (Global HIV/AIDS Medicine, 2008). The IUPAC name from fluconazole is 2-(2,4-difluorophenyl)-1,3-bis(1,2,4-triazol-1-yl)propan-2-ol.

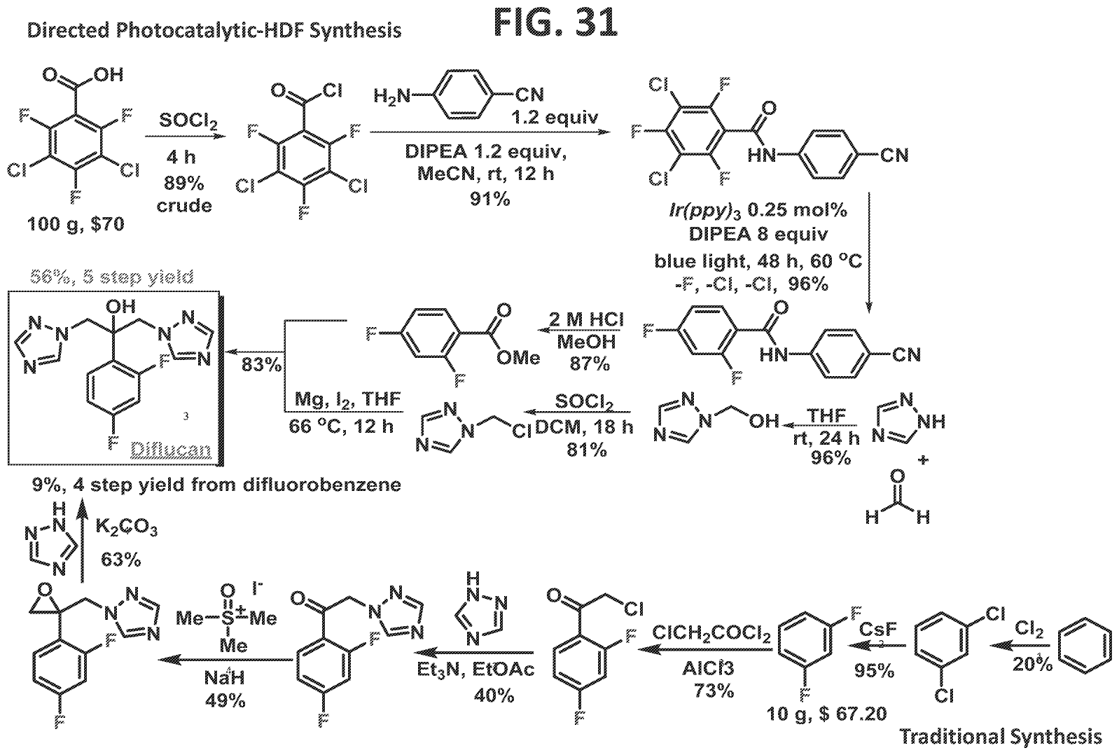

FIG. 31 illustrates synthesis of fluconazole by the traditional prior art method (lower scheme, beginning on bottom right) and by the methods of the present disclosure (upper scheme, beginning on top left). The traditional method involves six steps if starting from benzene or four steps if starting from commercially available difluorobenzene. However, even when starting with difluorobenzene, the four step yield of fluconazole is 9%.

In contrast, the methods of the present disclosure use 3,5-dichloro-2,4,6-trifluorobenzoic acid as the starting material and include a five step synthesis that resulted, in one embodiment, in a 56% yield of fluconazole.

In addition, it is important to note that the cost for the prior art starting material of difluorobenzene is currently about $67 per 10 g, whereas the cost for the current method's starting material of 3,5-dichloro-2,4,6-trifluorobenzoyl chloride is about $70 for 100 g. Thus, the cost of starting material utilized in the present disclosure is an order of a magnitude lower than the starting material used in the prior art synthesis.

Sources for labeled prior art synthesis steps: (1) Faming Zhuanli Shenqing. (2) PCT App No. WO 2010/011812. (3) Chai et al., 2009; and Huaxi Yaoxue Zazhi. (4) U.S. Pat. No. 4,404,216 (1983). (5) Wang et al., 2014; and PCT App No. WO 2011/154700.

Supporting Information for Synthesis of Fluconazole:

Description of reaction shown in FIG. 40, Panel A: 3,5-dichloro-2,4,6-trifluorobenzoic acid (2.00 g, 8.20 mmol) was placed in a 50 mL round-bottomed flask and SOCl₂ (2.40 mL, 32.7 mmol) was added dropwise with stirring. The mixture was heated gently for 4 h at 70° C., and then cooled. A vacuum was applied, and the bath temperature was slowly increased to 50° C. to afford of 3,5-dichloro-2,4,6-trifluorobenzoyl chloride in 89% yield (1.92 g, 7.33 mmol) as a white solid.

Description of reaction shown in FIG. 40, Panel B: A solution of benzoyl chloride (1.00 g, 3.82 mmol) in MeCN (20 mL) was slowly added at 0° C. to a solution of 4-aminobenzonitrile (0.540 g, 4.58 mmol) and DIPEA (0.800 mL, 4.58 mmol) in MeCN (5 mL), which resulted in a white precipitate. After the reaction mixture was stirred for 12 hours at room temperature, the formed salt was removed by filtration and washed with THF (50 mL). All volatiles were removed in vacuo from the filtrate, after which 30 mL water was added, and the product was extracted with DCM (3×50 mL). The combined extract was washed with brine and dried over MgSO₄. Concentration under reduced pressure and subsequent purification by recrystallization (EtOAc/hexane) afforded N-benzoylaniline in 91% yield (1.20 g, 3.49 mmol) as a white solid.

Description of reaction shown in FIG. 40, Panel C: In an NMR tube capped with NMR septa (Ace glass, part no. 9096-25) was charged tris(2-phenyl pyridinato-C 2, N) Iridium(III) (Ir(ppy)₃) (0.250 mM, 1.00 mL in MeCN). 3,5-dichloro-N-(4-cyanophenyl)-2,4,6-trifluorobenzamide (34 mg, 0.100 mmol) and DIPEA (139 μL, 800 mmol) were added, and the reaction was degassed via Ar bubbling for 10 min at 0° C., to avoid evaporation of DIPEA and any other volatile starting materials, and then left under positive Ar pressure by removing the exit needle and then the argon supply needle. The tube was placed in a light bath (vide supra), and the lower portion of the tube was submerged under the water bath, which was maintained at 60° C. via the use of a thermostated heating mantle. The reaction was monitored by ¹⁹F NMR and GC-MS. After the complete consumption of starting material, the MeCN was removed via rotovap, and the residue was treated with deionized water (2 mL) and extracted with EtOAc (5×1 mL). The combined organic portions were dried over MgSO₄, filtered, and concentrated in vacuo. The crude material was purified by flash chromatography using hexane:ethyl acetate (0-40% EtOAc for 30 cv and ramped to 100% EtOAc for 30-50 cv and then held at 100% EtOAc 50-55 cv) on a 4 g silica column to afford of N-(4-cyanophenyl)-2,4-difluorobenzamide in 96% yield (24.0 mg, 0.0930 mmol) as a white solid.

Description of reaction shown in FIG. 40, Panel D: In a 25 mL round bottom flask equipped with a magnetic stir bar was charged with N-(4-cyanophenyl)-2,4-difluorobenzamide (100 mg, 0.387 mmol) and 12 M HCl (2 mL). The flask was placed in an oil bath and heated to 80° C. for 18 h. The reaction mixture was cooled to room temperature and MeOH (15 mL) was added. Then the mixture was stirred for 24 h at 70° C. After cooling the reaction mixture to room temperature, the solvent was removed under reduced pressure. This residue was poured into ice water (20 mL), and subsequently extracted with DCM (3×20 mL). After washing the combined organic phase with 15 mL brine, the organic layer was dried over MgSO₄, filtered, and the solvent was removed under reduced pressure. The crude material was purified by flash chromatography using hexane:ethyl acetate (0% for 12 cv, slowly ramped to 15% EtOAc for 15-25 cv and then ramped to 100% EtOAc for 25-35 cv, then held at 100% EtOAc 35 40 cv), on a 12 g silica column to afford methyl 2,4-difluorobenzoate in 87% yield (58.0 mg, 0.337 mmol) as a yellow oil.

Description of reaction shown in FIG. 40, Panel E: Triazole (1.00 g, 14.4 mmol) was placed in 100 mL flask. THF (40 mL) was added at 20° C. to give a clear solution. To this, formaldehyde (1.31 g, 37% aqueous solution) was added and stirred for 4 h to give a homogeneous solution. The solvent was removed under reduced pressure with a rotary evaporator, and the residue was dried in vacuo to afford (1H-1,2,4-triazol-1-yl)methanol in 96% yield (1.38 g, 13.9 mmol) as a white solid.

Description of reaction shown in FIG. 40, Panel F: In a 50 mL round-bottomed flask was charged (1H-1,2,4-triazol-1-yl)methanol (1.00 g, 10.1 mmol) in DCM (50 mL). SOCl₂ (2.93 mL, 40.4 mmol) was added dropwise with stirring. The mixture was heated gently for 4 h at 70° C., and then cooled. A vacuum was applied, and the bath temperature was slowly increased to 45° C. to afford 1-(chloromethyl)-1H-1,2,4-triazole in 81% yield (0.960 g, 8.21 mmol) as a white solid.

Description of reaction shown in FIG. 40, Panel G: An oven-dried, 100 ml round-bottom flask equipped with a reflux condenser was charged with magnesium turnings (26.0 mg, 1.07 mmol) and a crystal of iodine. The mixture was heated with a heat gun under vacuum, and then allowed to cool to room temperature. The flask was then charged with 15 mL of THF and refluxed for 15 min until the solution turned clear. The 1-(chloromethyl)-1H-1,2,4-triazole (100 mg, 0.854 mmol) was then added, and the mixture was refluxed for 1 h. The mixture was cooled to 0° C., and the methyl 2,4-difluorobenzoate (73.5 mg, 0.427 mmol) dissolved in 3 mL of THF was added dropwise. The reaction mixture was allowed to stir at 0° C. for 30 min, followed by room temperature for 1 h. The reaction was quenched with a gradual addition of 6 mL of saturated ammonium chloride solution. The layers were separated, and the aqueous phase was extracted with Et₂O (3×30 mL). The organic layers were combined, dried over MgSO₄, and concentrated in vacuo. The crude material was purified by flash chromatography using hexane: ethyl acetate (0-35% EtOAc for 20 cv and ramped to 100% EtOAc for 20-35 cv and then held at 100% EtOAc 35-45 cv) on a 12 g silica column to afford 2-(2,4-difluorophenyl)-1,3-di(1H-1,2,4-triazol-1-yl)propan-2-ol in 83% yield (108 mg, 0.352 mmol) as brown solid.

### Example 5—Synthesis of Melperone

Melperone is an atypical antipsychotic of the butyrophenone chemical class, making it structurally related to the typical antipsychotic haloperidol. Melperone has been used clinically since the 1960s. It has been well established in the treatment of confusion, anxiety, restlessness (particularly in the elderly), and schizophrenia, as it is known to be well-tolerated with an excellent safety profile. In addition, melperone has recently been studied as a treatment of psychosis related to Parkinson's disease. The IUPAC name for melperone is 1-(4-fluorophenyl)-4-(4-methylpiperidin-1-yl)butan-1-one.

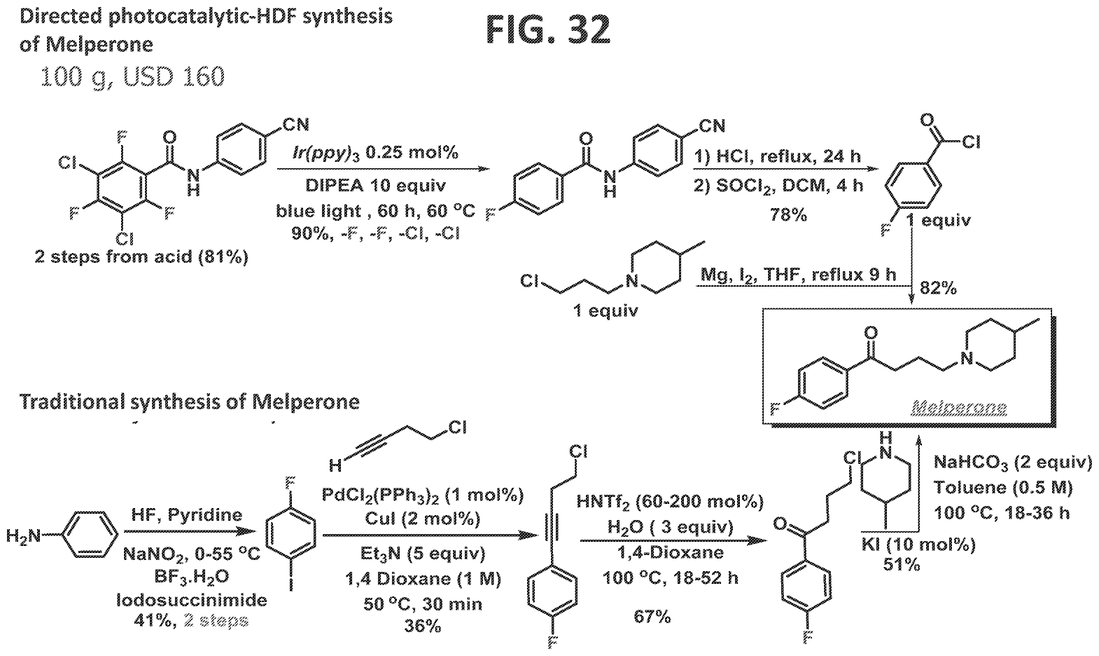

FIG. 32 illustrates synthesis of melperone by the traditional prior art method (lower scheme, beginning on the lower left) and by the methods of the present disclosure (upper scheme, beginning on upper left).

The prior art methods utilize aniline as starting material and involve four steps, with an overall yield near 5%. In contrast, the current method utilizes 2-(3,5-dichloro-2,4,6-trifluorophenyl)-N-(4-cyanophenyl)acetamide as a starting material and involves three steps, with a yield of 58%, or 47% starting with the corresponding acid.

Sources for labeled prior art synthesis steps: (1) Olah et al., 1979; and Prakash et al., 2012. (2) Leyva-Pérez et al., 2014.

Supporting Information for Synthesis of Melperone:

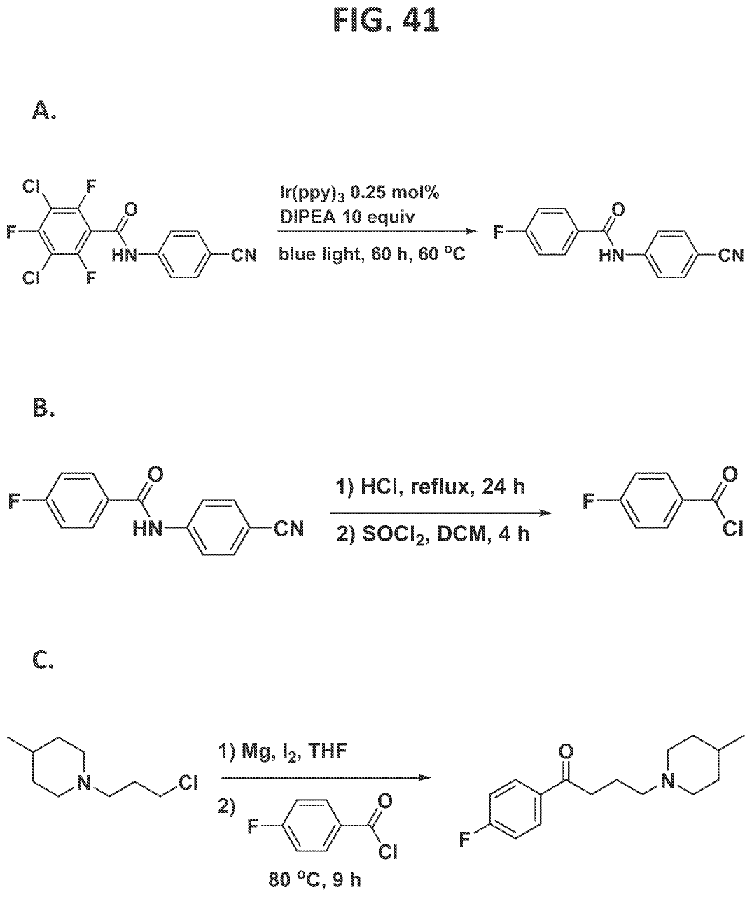

Description of reaction shown in FIG. 41, Panel A: In an NMR tube capped with NMR septa (Ace glass, part no. 9096-25) was charged tris(2-phenyl pyridinato-C 2, N) Iridium(III) (Ir(ppy)₃) (0.250 mM, 1.00 mL in MeCN). 3,5-dichloro-N-(4-cyanophenyl)-2,4,6-trifluorobenzamide (34.0 mg, 0.100 mmol, as described above) and DIPEA (174 pi, 1.00 mmol) were added, and the reaction was degassed via Ar bubbling for 10 min, at 0° C., to avoid evaporation of DIPEA and any other volatile starting materials, and then left under positive Ar pressure by removing the exit needle and then the argon supply needle. The tube was placed in a light bath (vide supra), and the lower portion of the tube was submerged under the water bath which was maintained at 60° C. via the use of a thermostated heating mantle. The reaction was monitored by ¹⁹F NMR and GC-MS. After the complete consumption of starting material, the MeCN was removed via rotovap, and the residue was treated with deionized water (2 mL) and extracted with EtOAc (5×1 mL). The combined organic portions were dried with over MgSO₄, filtered, and concentrated in vacuo. The crude material was purified by flash chromatography using hexane:ethyl acetate (0-45% EtOAc for 25 cv and ramped to 100% EtOAc for 25-45 cv and then held at 100% EtOAc 45-50 cv) on a 4 g silica column to afford N-(4-cyanophenyl)-4-fluorobenzamide in 91% yield (22.0 mg, 0.0916 mmol) as yellow solid.

Description of reaction shown in FIG. 41, Panel B: N-(4-cyanophenyl)-4-fluorobenzamide (100 mg, 0.416 mmol) was taken in an 25 mL round-bottom flask which was connected through a condenser under a Ar atmosphere, and 12 M HCl (2 mL) was added. Then the mixture was stirred for 24 h at 110° C. followed by cooling. Water (10 mL) was added. The aqueous layer was extracted with EtOAc (3×30 mL). The combined organic layers were washed with brine (10 mL), dried over MgSO₄, filtered, and concentrated in vacuo to afford the 4-fluorobenzoic acid. Then, a 50 mL round-bottomed flask was charged 4-fluorobenzoic acid in DCM (3 mL). SOCl₂ (2.93 mL, 40.4 mmol) was added dropwise with stirring. The mixture was heated gently for 4 h at 70° C., and then cooled. A vacuum was applied, and the bath temperature was slowly increased to 45° C. to afford 4-fluorobenzoyl chloride in 78% yield (51.0 mg, 0.322 mmol) as orange oil.

Description of reaction shown in FIG. 41, Panel C: An oven-dried, 100 mL round-bottom flask equipped with a reflux condenser was charged with magnesium turnings (83.2 mg, 3.42 mmol) and a crystal of iodine. The mixture was heated with a heat gun under vacuum and allowed to cool to room temperature. The flask was then charged with 20 mL of THF and refluxed for 15 min until the solution turned clear. The 1-(3-chloropropyl)-4-methylpiperidine (200 mg, 1.14 mmol) was then added, and the mixture was refluxed for 1 h. The mixture was cooled to 0° C., and the 4-fluorobenzoyl chloride (181 mg, 1.14 mmol) dissolved in 4 mL of THF was added dropwise. The reaction mixture was allowed to stir at 0° C. for 25 min, followed by room temperature for 1 h. The reaction was quenched with a gradual addition of 15 mL of saturated ammonium chloride solution. The layers were separated, and the aqueous phase was extracted with EtOAc (3×30 mL). The organic layers were combined, dried over MgSO₄, and concentrated in vacuo, and the crude material was purified by flash chromatography using hexane:ethyl acetate (0-40% EtOAc for 19 cv and ramped to 100% EtOAc for 19-34 cv and then held at 100% EtOAc 34-40 cv) on a 12 g silica column to afford Melperone in 82% yield (248 mg, 0.942 mmol) as white solid.

### Example 6—Synthesis of Blonanserin

The methods of the present disclosure are also applicable to the synthesis of another atypical antipsychotic, blonanserin. Compared to many other antipsychotics, blonanserin has an improved tolerability profile and lacks side effects such as extrapyramidal symptoms, excessive sedation, and hypotension. As with many second-generation (atypical) antipsychotics, blonanserin is significantly more efficacious in the treatment of the negative symptoms of schizophrenia compared to first-generation (typical) antipsychotics such as haloperidol. The IUPAC name for blonanserin is 2-(4-ethylpiperazin-1-yl)-4-(4-fluorophenyl)-5,6,7,8,9,10-hexahydrocycloocta [b]pyridine.

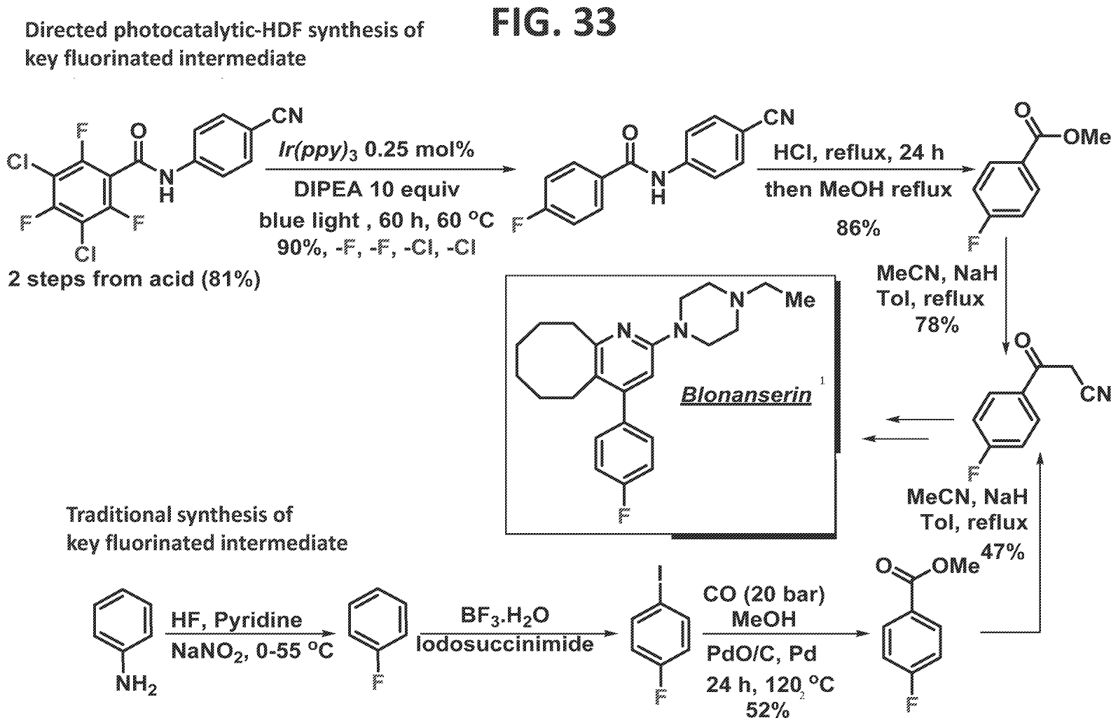

FIG. 33 illustrates synthesis of blonanserin by the traditional prior art method (lower scheme) and by the methods of the present disclosure (upper scheme).

The prior art methods utilize aniline as starting material and involve four steps to the key fluorinated intermediate, with a yield of 10% starting from aniline, and 47% yield in the final step. In contrast, the current method utilizes 2-(3,5-dichloro-2,4,6-trifluorophenyl)-N-(4-cyanophenyl)acetamide as a starting material and involves three steps to reach the key intermediate, with a yield of 60%.

Sources for labeled prior art synthesis steps: (1) Wang et al., 2014; PCT App Nos. WO 2011/154700 and WO 2003/076405. (2) Ziccarelli et al., 2016.

Supporting Information for Synthesis of Blonanserin:

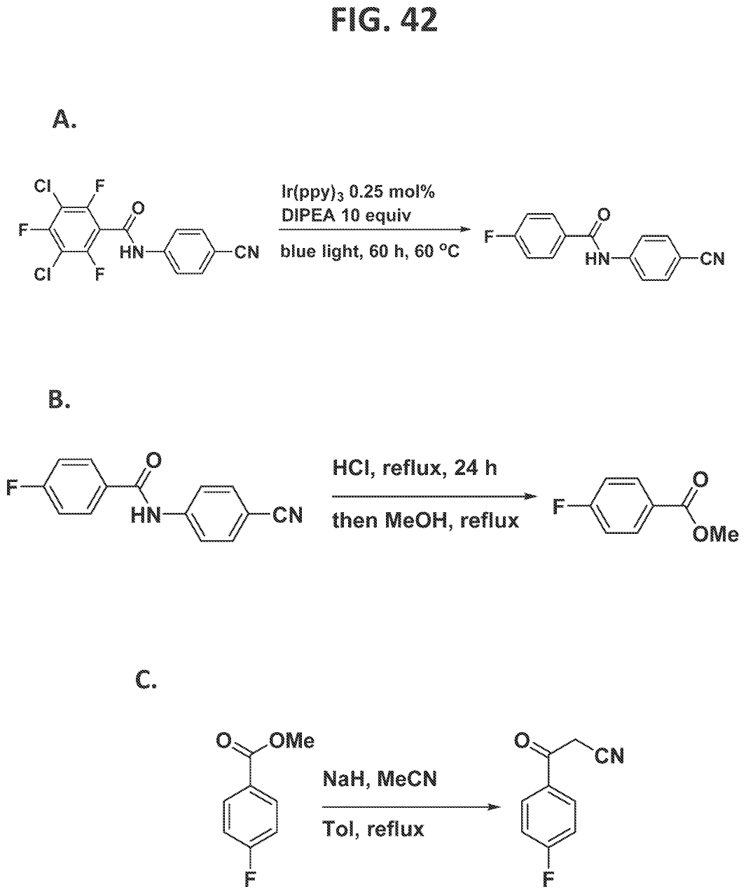

Description of reaction shown in FIG. 42, Panel A: The procedure was followed as described above.

Description of reaction shown in FIG. 42, Panel B: A 25 mL round bottom flask equipped with a magnetic stir bar was charged with N-(4-cyanophenyl)-4-fluorobenzamide (300 mg, 1.25 mmol) and 12 M HCl (5 mL). The flask was placed in an oil bath and heated to 80° C. for 24 h. The reaction mixture was cooled to room temperature, and MeOH (30 mL) was added. Then the mixture was stirred for 24 h at 70° C. After cooling the reaction mixture to room temperature, the solvent was removed under reduced pressure. This residue was poured into ice water (25 mL) and subsequently extracted with DCM (3×30 mL). After washing the combined organic phase with 20 mL brine, the organic layer was dried over MgSO₄, filtered, and the solvent was removed under reduced pressure. The crude material was purified by flash chromatography using hexane:ethyl acetate (0% for 12 cv, slowly ramped to 15% EtOAc for 15-20 cv and then ramped to 100% EtOAc for 20-36 cv, then held at 100% EtOAc 36 40 cv), on a 12 g silica column to afford methyl 4-fluorobenzoate in 86% yield (167 mg, 1.08 mmol) as a yellow oil.

Description of reaction shown in FIG. 42, Panel C: An oven-dried, 100 mL round-bottom flask equipped with a reflux condenser was charged with a solution of acetonitrile (170 μL, 3.24 mmol) in toluene (11 mL) and treated with methyl 4-fluorobenzoate (500 mg, 3.24 mmol), then sodium hydride (233 mg, 6.74 mmol, 60%) was added. The reaction mixture was refluxed until TLC indicated the total consumption of the ester. After cooling, the mixture was treated with ice-water (20 mL), acidified with 2 N HCl (to pH 2^{˜}3), and extracted with ethyl acetate (3×30 mL). The combined organic layer was dried over MgSO₄ and evaporated under reduced pressure to remove the solvent. The crude material was purified by flash chromatography using hexane:ethyl acetate (0-40% EtOAc for 25 cv and ramped to 100% EtOAc for 25-50 cv and then held at 100% EtOAc 50-55 cv) on a 12 g silica column to afford of 3-(4-fluorophenyl)-3-oxopropanenitrile in 78% yield (415 mg, 2.54 mmol) as a colorless oil.

### Example 7—Synthesis of Carbonic Anhydrase Inhibitor

CA inhibitors (CAIS) are clinically used mostly as antiglaucoma agents, as they are highly effective in reducing elevated intraocular pressure, after systemic administration of drugs such as acetazolamide, methazolamide, ethoxzolamide, or dichlorophenamide, or after topically administered sulfonamides, such as dorzolamide or brinzolamide. They are also useful for the treatment or prevention of other diseases, since different CA isozymes are widely distributed in higher vertebrates. The IUPAC name for the CAI illustrated in FIG. 31 is 2,3,5,6-tetrafluorophenyl-carboxamidosulfoamide.

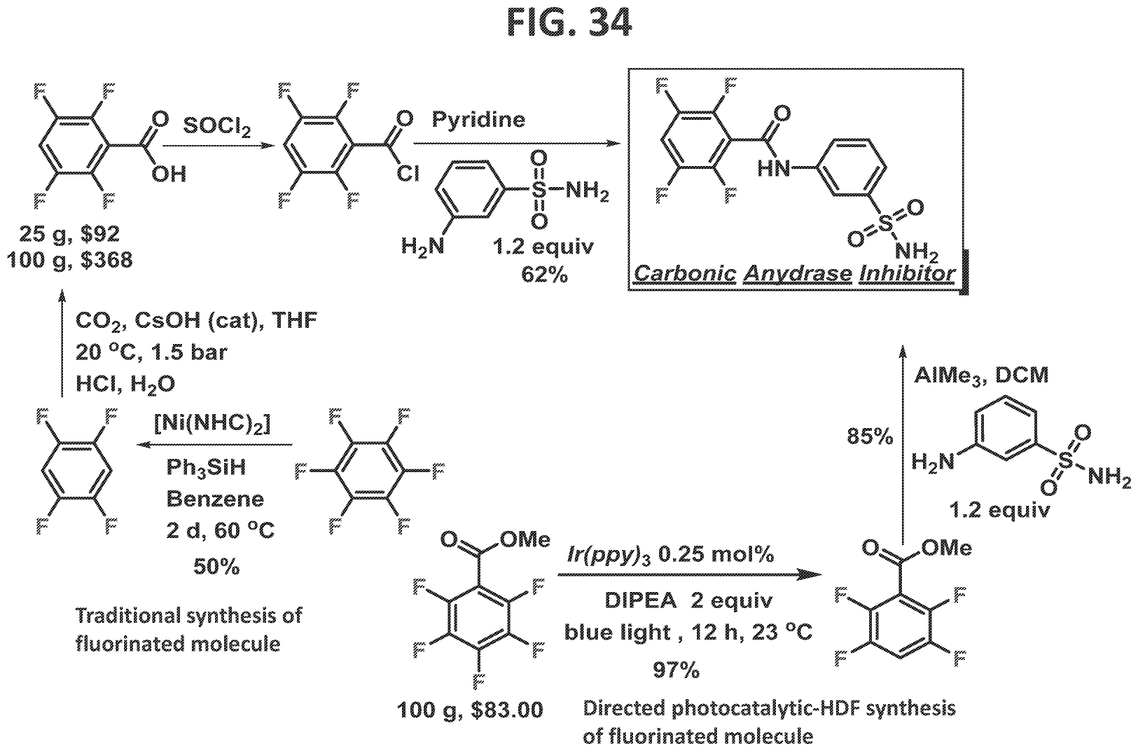

FIG. 34 illustrates synthesis of this carbonic anhydrase inhibitor by the traditional prior art method (upper scheme) and by the methods of the present disclosure (lower scheme). The traditional method utilizes hexafluorobenzene as starting material and involves four steps (Fischer et al., 2012; Boogaerts et al., 2010; and Pastorekova et al., 2005). In contrast, the current method involves two steps and utilizes the commercially available 2,3,4,5,6-pentafluorobenzoic acid (currently about $83 per 100 g) as starting material. The key steps here are the electronically controlled hydrodefluorination and the trans-amidation, which deliver the inhibitor in 82% yield from commercially available material.

Supporting Information for Synthesis of Carbonic Anhydrase Inhibitor:

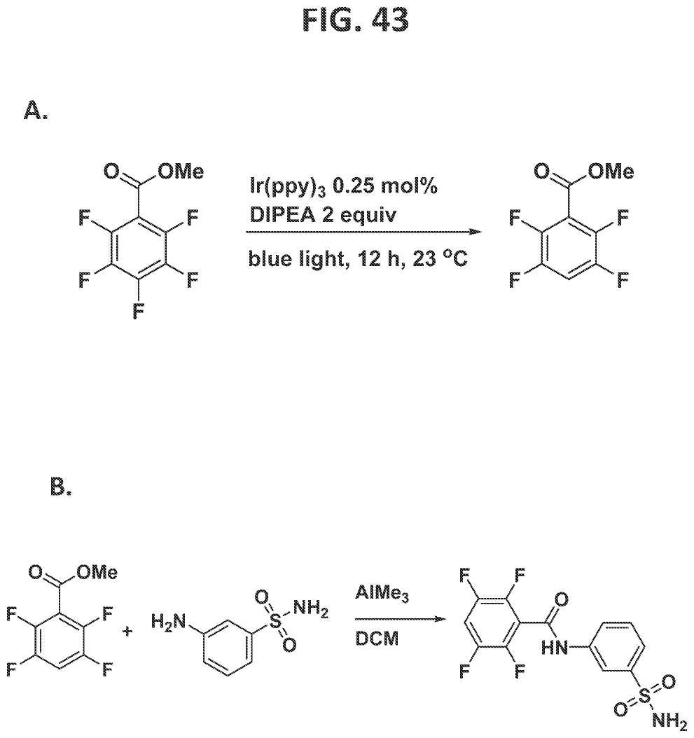

Description of reaction shown in FIG. 43, Panel A: In an NMR tube capped with NMR septa (Ace glass, part no. 9096-25) was charged tris(2-phenyl pyridinato-C 2, N) Iridium(III) (Ir(ppy)₃) (0.250 mM, 1.00 mL in MeCN), methyl 2,3,4,5,6-pentafluorobenzoate (23.0 mg, 0.100 mmol) and DIPEA (35 μL, 0.200 mmol) were added, and the reaction was degassed via Ar bubbling for 10 min, at 0° C., to avoid evaporation of DIPEA and any other volatile starting materials, and then left under positive Ar pressure by removing the exit needle and then the argon supply needle. The tube was placed in a light bath (vide supra), and the lower portion of the tube was submerged under the water bath which was maintained at 23° C. The reaction was monitored by ¹⁹F NMR and GC-MS. After the complete consumption of starting material, the MeCN was removed via rotovap, and the residue was treated with deionized water (2 mL) and extracted with EtOAc (5×1 mL). The combined organic portions were dried with over MgSO₄, filtered, and concentrated in vacuo. The crude material was purified by flash chromatography using hexane:EtOAc (0-50% EtOAc for 6 cv and ramped to 100% EtOAc for 6-26 cv and then held at 100% EtOAc 26-45 cv) on a 4 g silica column to afford of methyl 2,3,5,6-tetrafluorobenzoate in 97% yield (20.0 mg, 0.100 mmol) as a brown oil.

Description of reaction shown in FIG. 43, Panel B: To a cooled (0° C.) solution of 3-aminobenzenesulfonamide (99.0 mg, 0.576 mmol) in DCM (15 mL) was added a solution of AlMe₃ (2.4 equiv, 25 wt % in hexane). The reaction mixture was warmed to room temperature and continued to stir for 30 min until the gas evolution ceased. Then, methyl 2,3,5,6-tetrafluorobenzoate (50.0 mg, 0.240 mmol) was added, and the solution was refluxed for 12 h. The reaction mixture was cooled in an ice bath, and aq HCl (10%, 5 mL) was carefully added. The solution was extracted with DCM (3×15 mL), and the combined organic extracts were washed subsequently with a saturated aq solution of NaHCO₃ (15 mL) and brine (15 mL). Then, the organic layer was dried over MgSO₄. After filtration and removal of the solvent under vacuum, the crude material was purified by flash chromatography using hexane:EtOAc (0-50% EtOAc for 8 cv and ramped to 100% EtOAc for 8-42 cv and then held at 100% EtOAc 42-48 cv) on a 4 g silica column to afford 2,3,5,6-tetrafluoro-N-(3-sulfamoylphenyl)benzamide in 85% yield (70.0 mg, 0.201 mmol) as a brown solid.

### Example 8—Synthesis of Rufinamide

Rufinamide is a triazole derivative and an anticonvulsant medication utilized in the treatment of seizure disorders. Rufinamide was approved by the FDA in 2008 as adjunctive treatment of seizures associated with Lennox-Gastuat syndrome, a form of childhood epilepsy. The IUPAC name of rufinamide is 1-[(2,6-difluorophenyl)methyl]triazole-4-carboxamide.

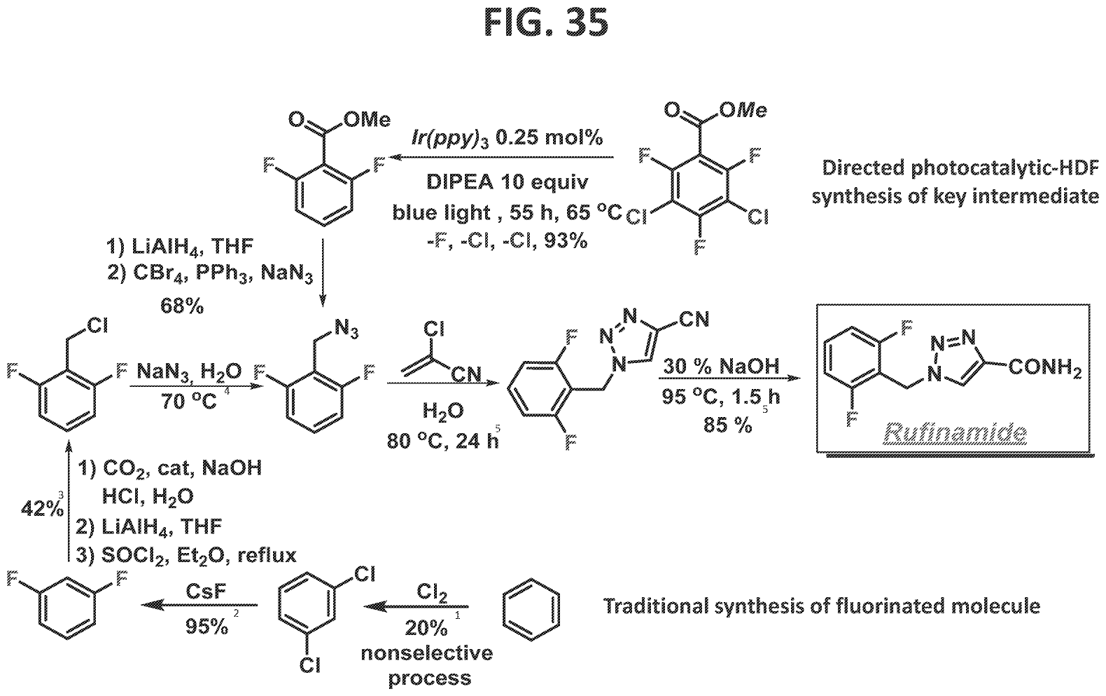

FIG. 35 illustrates synthesis of rufinamide by the traditional prior art method (lower scheme) and by the methods of the present disclosure (upper scheme).

The traditional method involves eight steps and utilizes benzene as starting material. In contrast, the current method (which actually utilize electronically controlled hydrodefluorination) uses methyl 3,5-dichloro-2,4,6-trifluorobenzoate as the starting material and involve a five-step telescoped synthesis with an overall yield of 54%.

Sources for labeled prior art synthesis steps: (1) Faming Zhuanli Shenqing. (2) PCT App No. WO 2010/011812. (3) Boogaerts et al., 2010; Wang et al., 2015; and Borukhova et al., 2016. (4) Taylor et al., 2017. (5) Wang et al., 2014; and PCT App No. 2011/154700.

Supporting Information for Synthesis of Rufinamide:

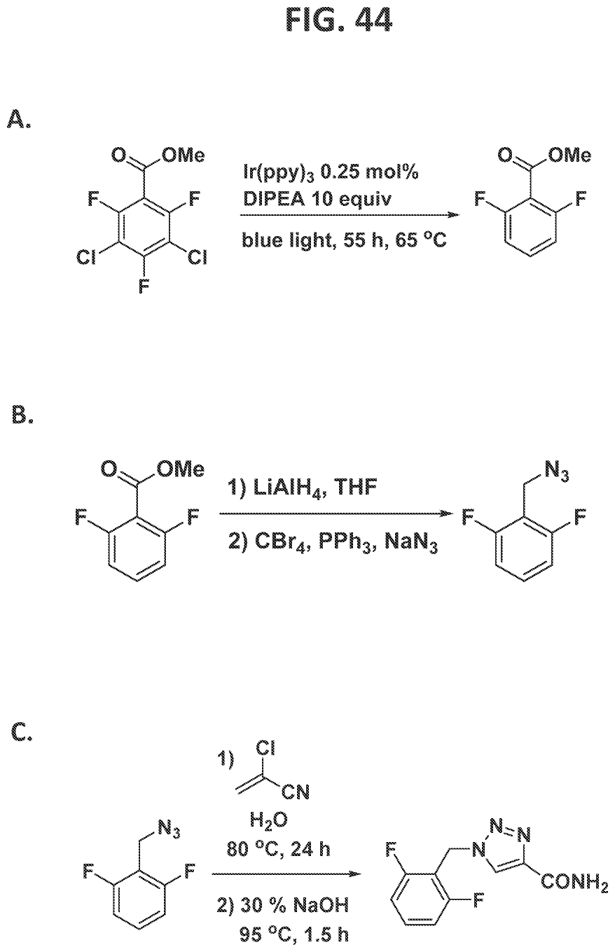

Description of reaction shown in FIG. 44, Panel A: In an NMR tube capped with NMR septa (Ace glass, part no. 9096-25) was charged tris(2-phenyl pyridinato-C 2, N) Iridium(III) (Ir(ppy)₃) (0.250 mM, 1.00 mL in MeCN). Methyl 3,5-dichloro-2,4,6-trifluorobenzoate (26.0 mg, 0.100 mmol) and DIPEA (174 μL, 1.00 mmol) were added, and the reaction was degassed via Ar bubbling for 10 min, at 0° C., to avoid evaporation of DIPEA and any other volatile starting materials, and then left under positive Ar pressure by removing the exit needle and then the argon supply needle. The tube was placed in a light bath (vide supra), and the lower portion of the tube was submerged under the water bath, which was maintained at 65° C. via the use of a thermostated heating mantle. The reaction was monitored by ¹⁹F NMR and GC-MS. After the complete consumption of starting material, the MeCN was removed via rotovap, and the residue was treated with deionized water (2 mL) and extracted with EtOAc (5×1 mL). The combined organic portions were dried over MgSO₄, filtered, and concentrated in vacuo. The crude material was purified by flash chromatography using hexane:EtOAc (0-40% EtOAc for 8 cv and ramped to 100% EtOAc for 8-20 cv and then held at 100% EtOAc 20-33 cv) on a 4 g silica column to afford methyl 2,6-difluorobenzoate in 93% yield (16.0 mg, 0.0930 mmol) as a colorless oil.

Description of reaction shown in FIG. 44, Panel B: In a 50 mL round bottom flask equipped with a magnetic stir bar was charged with a solution of methyl 2,6-difluorobenzoate (500 mg, 2.90 mmol) in THF (15 mL) was added dropwise to a solution of LiAlH₄ (200 mg, 5.27 mmol) in THF (15 mL) at 0° C. The resulting solution was allowed to warm to room temperature and stirred for 16 h, and the reaction was quenched by the addition of aq 1 M KOH (15 mL) dropwise at 0° C. Et₂O (30 mL) was then added, and the mixture stirred for 30 min at room temperature, the layers separated, and the aqueous layer extracted with Et₂O (2×30 mL). The combined organic layers were washed with aq 1 M HCl (30 mL), then brine (30 mL), dried over MgSO₄, and concentrated in vacuo to give the alcohol as a colorless oil, which was used without further purification. A solution of alcohol (400 mg, 2.77 mmol) in 15 mL of Et₂O containing triphenylphosphine (728 mg, 2.77 mmol) and carbon tetrabromide (918 mg, 2.77 mmol) was stirred at 25° C. for 2 h, filtered, and concentrated in vacuo. The crude material was purified by flash chromatography using hexane:EtOAc (0-40% EtOAc for 11 cv and ramped to 100% EtOAc for 11-30 cv and then held at 100% EtOAc 30-40 cv) on a 12 g silica column to afford the alkyl bromide. In a 50 mL round bottom flask equipped with a magnetic stir bar was charged with alkyl bromide (450 mg, 2.18 mmol) and NaN₃ (283 mg, 4.36 mmol) in DMF (3 mL) and was stirred at 50° C. for 24 h. Then the mixture was partitioned between water (50 mL) and Et₂O (3×50 mL). The organic phase was washed with water (2×10 mL), collected, and dried over MgSO₄. The solvent was removed under reduced pressure to afford 2-(azidomethyl)-1,3-difluorobenzene as colorless oil. The three steps in the preparation afforded 2-(azidomethyl)-1,3-difluorobenzene in 68% yield (330 mg, 1.95 mmol).

Description of reaction shown in FIG. 44, Panel C: In a 50 mL round bottom flask equipped with a magnetic stir bar was charged with 2-(azidomethyl)-1,3-difluorobenzene (200 mg, 1.18 mmol) and 2-chloroacrylonitrile (200 μL, 2.28 mmol) in H₂O (10 mL). The reaction mixture was stirred continuously at 80° C. for 24 h. After cooling, 30% NaOH (5 mL) was added, and the mixture was refluxed for 1.5 h. The residue was treated with deionized water (50 mL) and extracted with EtOAc (3×40 mL). The combined organic portions were dried over MgSO₄, filtered, and concentrated in vacuo to afford 1-(2,6-difluorobenzyl)-1H-1,2,3-triazole-4-carbonitrile in 85% yield (240 mg, 1 mmol) as a white solid.

### Example 9—Synthesis of Elvitegravir

Elvitegravir is a modified quinolone antibiotic with activity against human immunodeficiency virus 1 (HIV-1) infection. Elvitegravir is an inhibitor of viral integrase and retains activity against integrase mutants that are resistant to Raltegravir. The IUPAC name of elvitegravir is 6-[(3-chloro-2-fluorophenyl)methyl]-1-[(25)-1-hydroxy-3-methylbutan-2-yl]-7-methoxy-4-oxoquinoline-3-carboxylic acid.

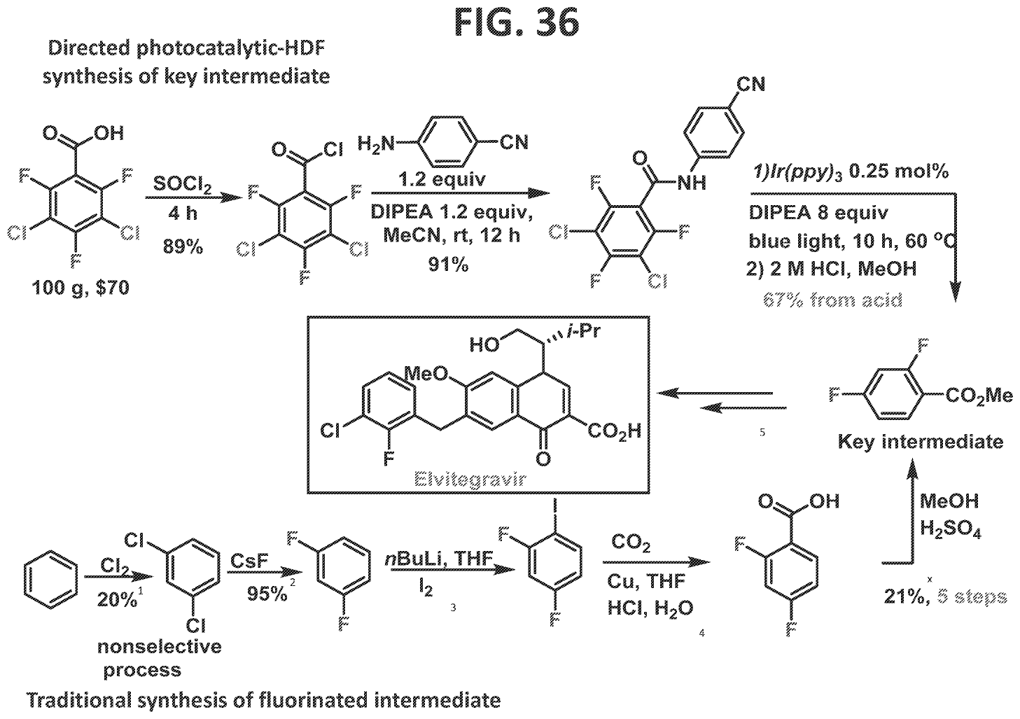

FIG. 36 illustrates synthesis of elvitegravir by the traditional prior art method (lower scheme) and by the methods of the present disclosure (upper scheme).

The traditional method involves five steps (21% yield) and utilizes benzene as starting material to arrive at the key intermediate en route to the drug. In contrast, the current method uses 3,5-dichloro-2,4,6-trifluorobenzoic acid as the starting material and includes a four step synthesis in 67% overall yield.

Sources for labeled prior art synthesis steps: (1) Faming Zhuanli Shenqing. (2) PCT App No. WO 2010/011812. (3) Mongin et al., 2001. (4) Ebert et al., 2005. (5) PCT App No. WO 2012/119046.

Supporting Information for Synthesis of Elvitegravir:

The above described procedures were used.

### Example 10—Synthesis of Dolutegravir

Dolutegravir is an orally bioavailable integrase strand-transfer inhibitor (INSTI), with activity against HIV-1 infection. The IUPAC name of dolutegravir is (4R,12aS)-N-[(2,4-difluorophenyl)methyl]-7-hydroxy-4-methyl-6,8-dioxo-3,4,12,12a-tetra hydro-2H-pyrido[5,6]pyrazino[2,6-b][1,3]oxazine-9-carboxamide.

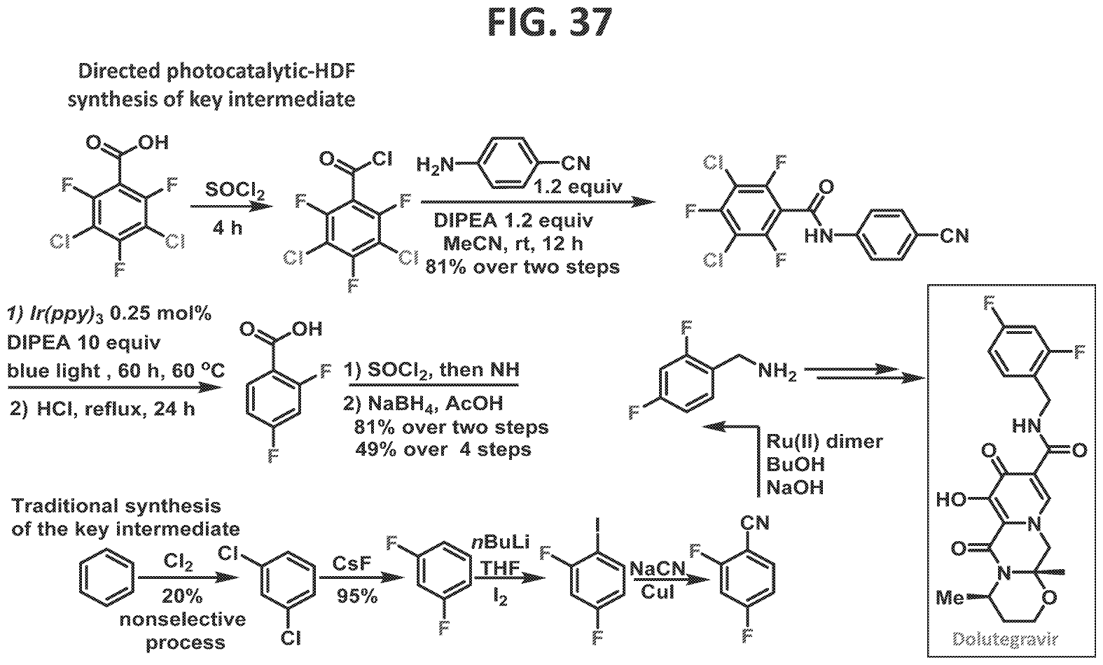

FIG. 37 illustrates synthesis of dolutegravir by the traditional prior art method (lower scheme) and by the methods of the present disclosure (upper scheme).

The traditional method involves five steps to arrive at the key difluorinated intermediate and utilizes benzene as starting material (PCT App No. WO 2010/011812). In contrast, the current method uses 3,5-dichloro-2,4,6-trifluorobenzoic acid as the starting material and include a six step telescoped synthesis, resulting in 49% overall yield.

Supporting Information for Synthesis of Dolutegravir:

The preparations of benzoyl chloride and the amide were described above.

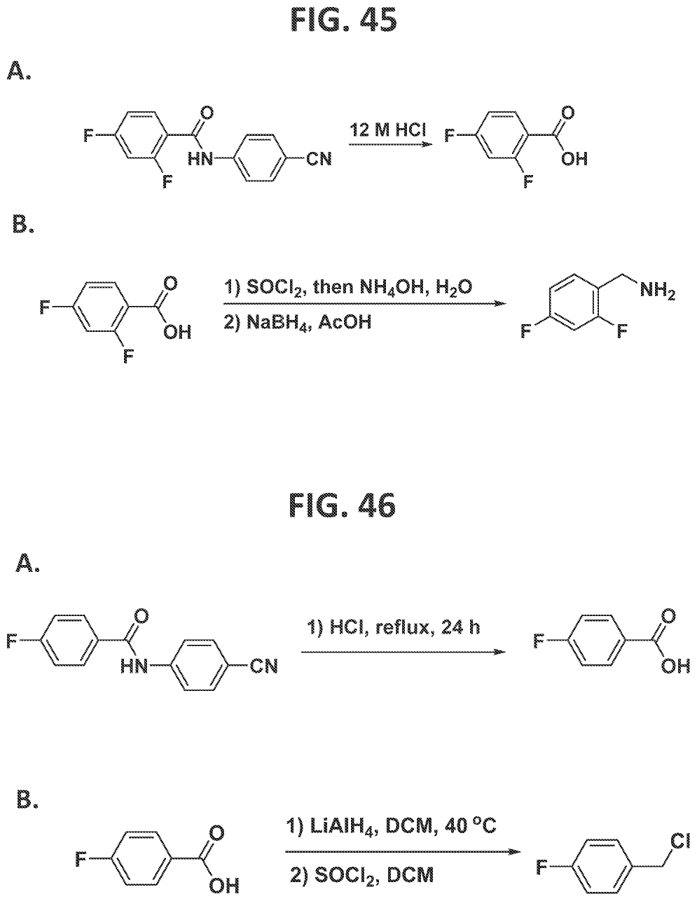

Description of reaction shown in FIG. 45, Panel A: In a 25 mL round bottom flask equipped with a magnetic stir bar was charged with N-(4-cyanophenyl)-2,4-difluorobenzamide (100 mg, 0.387 mmol) and 12 M HCl (2 mL). The flask was placed in an oil bath and heated to 80° C. for 18 h. Then the mixture was partitioned between water (5 mL) and Et₂O (3×10 mL). The combined organic portions were dried over MgSO₄, filtered, and concentrated in vacuo to afford 2,4-difluorobenzoic acid in 98% yield (60 mg, 0.386 mmol) as yellow oil.

Description of reaction shown in FIG. 45, Panel B: 2,4-difluorobenzoic acid (100 mg, 0.632 mmol) was placed in a 50 mL round-bottomed flask, and SOCl₂ (183 μL, 2.53 mmol) was added dropwise with stirring. The mixture was heated gently for 4 h at 70° C. and then cooled. A vacuum was applied, and the bath temperature was slowly increased to 50° C. to remove the volatile reagents. The residue was treated with NH₄OH (4 mL) and water (2 mL) in THF (1 mL) with continuous stirring for 3 h. Then deionized water (20 mL) was added and extracted with EtOAc (3×20 mL). The combined organic portions were dried over MgSO₄, filtered, and concentrated in vacuo. A 50 mL round bottom flask equipped with a magnetic stir bar was charged with NaBH₄ (60 mg, 1.58 mmol) suspended in THF (15 mL), then cooled down to 0° C. with an ice-water bath. A solution of benzamide in THF (1 mL) was added dropwise over a period of 15 minutes. The mixture was stirred at 0° C. for 1 h and stirred at room temperature for one day. The solution was extracted with Et₂O (3×20 mL), and the combined organic extracts were washed with brine (15 mL) consequently. Then the organic layer was dried over MgSO₄. After filtration and removal of the solvent under vacuum, the crude material was purified by flash chromatography using hexane:EtOAc (0-50% EtOAc for 8 cv and ramped to 100% EtOAc for 8-35 cv and then held at 100% EtOAc 35-50 cv) on a 4 g silica column to afford (2,4-difluorophenyl)methanamine in 81% yield (72.0 mg, 0.503 mmol) as a yellow solid.

### Example 11—Synthesis of Darapladib

Darapladib is a substituted pyrimidone with inhibitory activity towards lipoprotein-associated phospholipase-A2 (Lp-PLA2), an important regulator of lipid metabolism and inflammation that circulates with lipoprotein particles and is carried into the arterial wall with low-density lipoprotein particles during the progression of atherosclerosis. The IUPAC name for darapladib is N-[2-(diethylamino)ethyl]-2-[2-[(4-fluorophenyl)methylsulfanyl]-4-oxo-6,7-dihydro-5H-cyclopenta[d]pyrimidin-1-yl]-N-[[4-[4-(trifluoromethyl)phenyl]phenyl]methyl] acetamide.

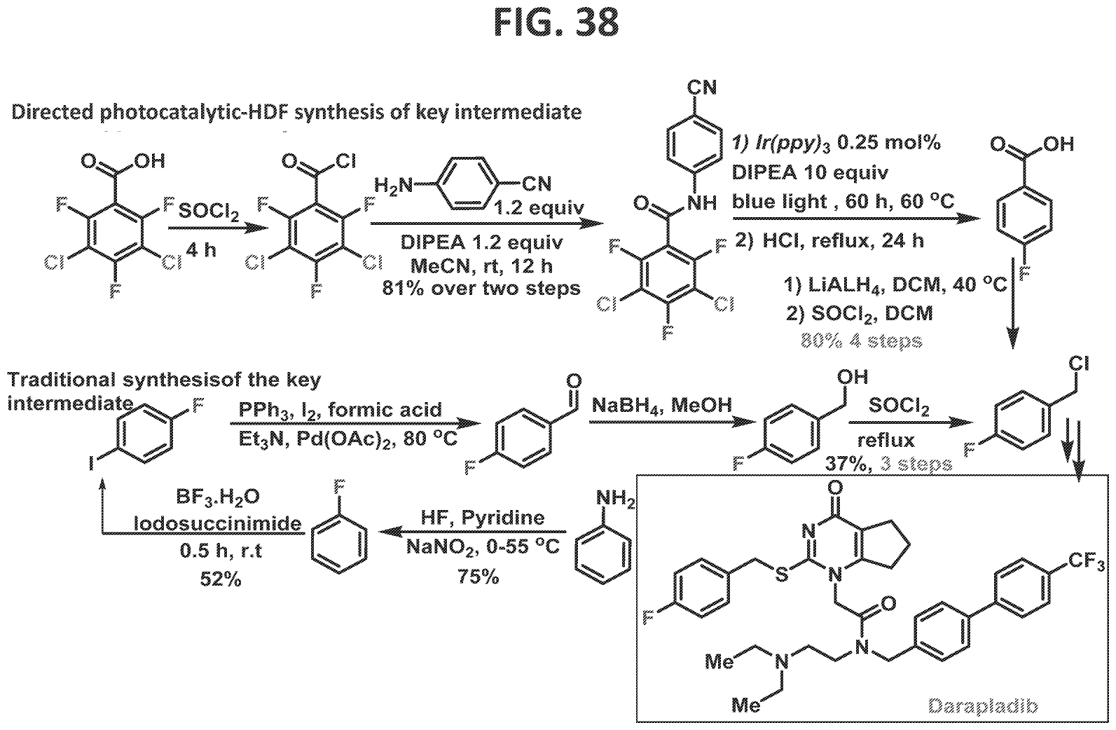

FIG. 38 illustrates synthesis of darapladib by the traditional prior art method (lower scheme) and by the methods of the present disclosure (upper scheme).

The traditional method involves five steps and utilizes aniline as starting material, providing the key fluorinated intermediate in 14% yield (Boyd et al., 2000). In contrast, the current method uses 3,5-dichloro-2,4,6-trifluorobenzoic acid as the starting material and includes a six step telescoped synthesis that provides the key fluorinated arene in 65% yield.

Supporting Information for Synthesis of Darapladib:

The preparations of benzoyl chloride and the amide were described above.

Description of reaction shown in FIG. 46, Panel A: In a 25 mL round bottom flask equipped with a magnetic stir bar was charged with N-(4-cyanophenyl)-4-fluorobenzamide (100 mg, 0.416 mmol) and 12 M HCl (2 mL). The flask was placed in an oil bath and heated to 120° C. for 24 h. Then the mixture was partitioned between water (5 mL) and Et₂O (3×20 mL). The combined organic portions were dried over MgSO₄, filtered, and concentrated in vacuo to afford 4-fluorobenzoic acid in 98% yield (57 mg, 0.407 mmol) as yellow liquid.

Description of reaction shown in FIG. 46, Panel B: In a 25 mL round bottom flask equipped with a magnetic stir bar was charged LiAlH₄ (29.0 mg, 0.771 mmol) in DCM, cooled down with an ice bath, and then a solution (DCM) of the acid resulting from the previous step was added dropwise (90 mg, 0.642 mmol). After the addition, the mixture was allowed to warm to room temperature and stirred for an additional 18 hours. After the reaction was complete, it was quenched with aqueous NaOH solution (4 M, 10 mL) and filtered through Celite, then rinsed twice with DCM (2×30 mL). The filtrate was dried over MgSO₄, filtered, and concentrated in vacuo, and the crude residue was used in the next step without any further purification. The alcohol resulting from the previous step was placed in a 50 mL round-bottomed flask, and SOCl₂ (2.00 mL, 27.5 mmol) was added dropwise with stirring. The mixture was heated gently for 12 h at 40° C. After the complete consumption of starting material, water (10 mL) was poured in the flask, then solution was extracted with Et₂O (3×20 mL), and the combined organic extracts were washed with brine (15 mL) consequently. Then the organic layer was dried over MgSO₄. After filtration and removal of the solvent under vacuum, the crude material was purified by flash chromatography using hexane:ethyl acetate (0% for 10 cv, slowly ramped to 16% EtOAc for 16-24 cv and then ramped to 100% EtOAc for 24-40 cv, then held at 100% EtOAc 40 45 cv), on a 4 g silica column to afford 1-(chloromethyl)-4-fluorobenzene in 96% yield (90.0 mg, 0.620 mmol) as a colorless oil.

### Example 12—Synthesis of Oxo-Ezogabine Analog

Ezogabine, which is known as retigabine in Europe, is a unique anticonvulsant used largely as an adjunctive agent in the treatment of partial seizures.

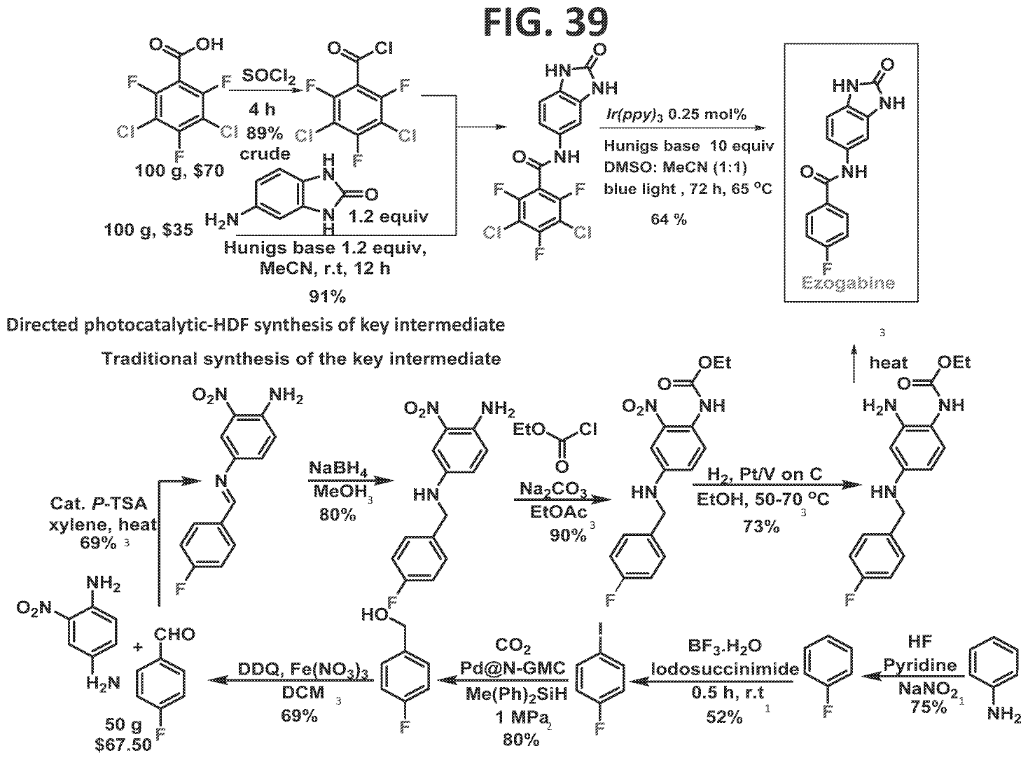

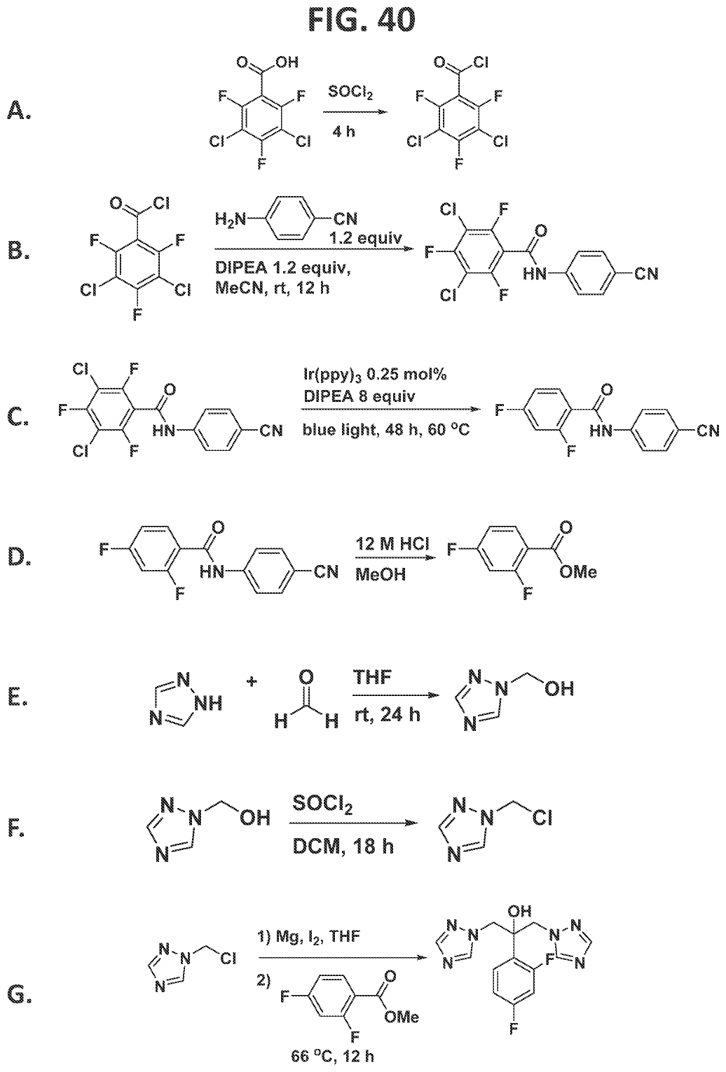

FIG. 39 illustrates synthesis of oxo-ezogabine analog by the traditional prior art method (lower scheme) and by the methods of the present disclosure (upper scheme).

The traditional method involves nine steps and utilizes aniline as starting material. In contrast, the methods of the present disclosure uses either 3,5-dichloro-2,4,6-trifluorobenzoyl chloride or 3,5-dichloro-2,4,6-trifluorobenzoic acid as the starting material and include a two or three step synthesis, respectively.

Sources for labeled prior art synthesis steps: (1) Olah et al., 1979; and Prakash et al., 2012. (2) Molla et al., 2016. (3) PCT App No. WO 2011/089126; and Wang et al., 2014.

Supporting Information for Synthesis of Oxo-Ezogabine Analog:

Oxo-Ezogabine analog of benzoyl chloride described above.

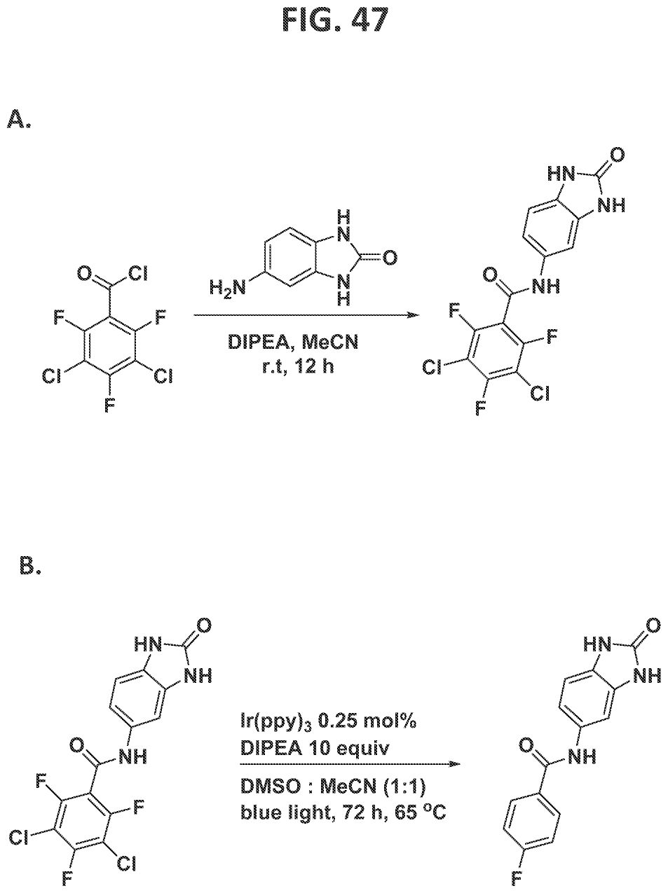

Description of reaction shown in FIG. 47, Panel A: A solution of benzoyl chloride (1.00 g, 3.82 mmol) in MeCN (20 mL) was slowly added at 0° C. to a solution of 5-amino-1,3-dihydro-2H-benzo[d]imidazol-2-one (0.682 g, 4.58 mmol) and DIPEA (0.800 mL, 4.58 mmol) in MeCN (5 mL), which resulted in a white precipitate. After the reaction mixture was stirred for 12 hours at room temperature, the formed salt was removed by filtration and washed with THF (50 mL). All volatiles were removed in vacuo from the filtrate, after which 30 mL water added, and it was extracted with DCM (3×30 mL). The extract was washed with brine and dried over MgSO₄. Concentration under reduced pressure and subsequent purification by recrystalization (EtOAc/hexane) afforded 3,5-dichloro-2,4,6-trifluoro-N-(2-oxo-2,3-dihydro-1H-benzo[d]imidazol-5-yl)benzamide in 91% yield (1.30 g, 3.46 mmol) as a white solid.

Description of reaction shown in FIG. 47, Panel B: In an NMR tube capped with NMR septa (Ace glass, part no. 9096-25) was charged tris(2-phenyl pyridinato-C 2, N) Iridium(III) (Ir(ppy)₃) (0.250 mM, 0.500 mL in MeCN). 3,5-dichloro-2,4,6-trifluoro-N-(2-oxo-2,3-dihydro-1H-benzo[d]imidazol-5-yl)benzamide (37.0 mg, 0.100 mmol) in DMSO (0.500 mL) and DIPEA (174 μL, 1.00 mmol) were added, and the reaction was degassed via Ar bubbling for 10 min at 0° C., to avoid evaporation of DIPEA and any other volatile starting materials, and then left under positive Ar pressure by removing the exit needle and then the argon supply needle. The tube was placed in a light bath (vide supra), and the lower portion of the tube was submerged under the water bath, which was maintained at 65° C. via the use of a thermostated heating mantle. The reaction was monitored by ¹⁹F NMR and GC-MS. After the complete consumption of starting material, the MeCN was removed via rotovap, and the residue was treated with deionized water (2 mL) and extracted with EtOAc (5×1 mL). The combined organic portions were dried over MgSO₄, filtered, and concentrated in vacuo. The crude material was purified by flash chromatography using hexane:ethyl acetate (0% for 18 cv, slowly ramped to 20% EtOAc for 18-26 cv and then ramped to 100% EtOAc for 26-33 cv, then held at 100% EtOAc 38 46 cv), on a 4 g silica column to afford 4-fluoro-N-(2-oxo-2,3-dihydro-1H-benzo[d]imidazol-5-yl)benzamide in yield of 64% (46.0 mg, 0.169 mmol) as a black solid.

### Example 13—Synthesis of Ezogabine

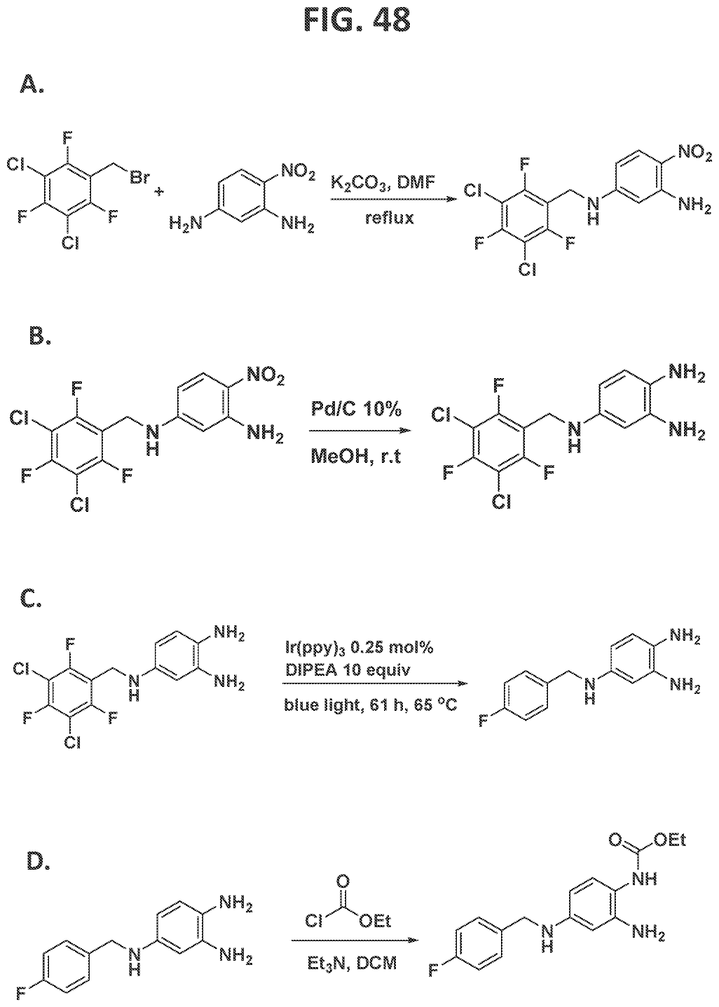

Description of reaction shown in FIG. 48, Panel A: An oven-dried, 100 mL round-bottom flask equipped with a reflux condenser was charged with K₂CO₃ (2.37 g, 17.1 mmol) in DMF (10 mL), to which was added substituted aniline (0.524 g, 3.42 mmol) followed by the polyhalogenated bromide (1.00 g, 3.42 mmol). The resulting mixture was placed in a heating bath and heated to reflux for 12 h, then cooled to room temperature and diluted with water (150 mL) and EtOAc (150 mL). The organic layer was separated, and the aqueous layer was extracted with EtOAc (2×150 mL). The combined organic extractions were washed with brine (150 mL), dried over MgSO₄, and then were concentrated under reduced pressure to afford the crude product. The crude material was purified by flash chromatography using hexane:ethyl acetate (0% for 10 cv, slowly ramped to 20% EtOAc for 20-30 cv, and then ramped to 100% EtOAc for 30-45 cv, then held at 100% EtOAc 45 55 cv), on a 12 g silica column to afford N1-(3,5-dichloro-2,4,6-trifluorobenzyl)-4-nitrobenzene-1,3-diamine in 64% yield (0.800 g, 2.19 mmol) as a yellow solid.

Description of reaction shown in FIG. 48, Panel B: An oven-dried, 100 mL round-bottom flask equipped with a reflux condenser was charged with N1-(3,5-dichloro-2,4,6-trifluorobenzyl)-4-nitrobenzene-1,3-diamine (200 mg, 0.547 mmol) in MeOH (10 mL) was hydrogenated in the presence of 10% Pd—C catalyst (1 g) and H₂ (balloon), at room temperature with stirring overnight. The mixture was filtered through celite and evaporated in vacuo to afford the crude product. The crude material was purified by flash chromatography using hexane:ethyl acetate (0-45% EtOAc for 22 cv and ramped to 100% EtOAc for 22-45 cv and then held at 100% EtOAc 45-50 cv) on a 12 g silica column to afford N4-(3,5-dichloro-2,4,6-trifluorobenzyl)benzene-1,2,4-triamine in 81% yield (148 mg, 0.442 mmol) as black solid.

Description of reaction shown in FIG. 48, Panel C: In an NMR tube capped with NMR septa (Ace glass, part no. 9096-25) was charged tris(2-phenyl pyridinato-C 2, N) Iridium(III) (Ir(ppy)₃) (0.250 mM, 1.00 mL in MeCN). N4-(3,5-dichloro-2,4,6-trifluorobenzyl)benzene-1,2,4-triamine (34.0 mg, 0.100 mmol, as described above) and DIPEA (174 μL, 1.00 mmol) were added, and the reaction was degassed via Ar bubbling for 10 min at 0° C., to avoid evaporation of DIPEA and any other volatile starting materials, and then left under positive Ar pressure by removing the exit needle and then the argon supply needle. The tube was placed in a light bath (vide supra), and the lower portion of the tube was submerged under the water bath, which was maintained at 60° C. via the use of a thermostated heating mantle. The reaction was monitored by ¹⁹F NMR and GC-MS. After the complete consumption of starting material, the MeCN was removed via rotovap, and the residue was treated with deionized water (2 mL) and extracted with EtOAc (5×1 mL). The combined organic portions were dried with over MgSO₄, filtered, and concentrated in vacuo. The crude material was purified by flash chromatography using hexane:ethyl acetate (0-45% EtOAc for 30 cv and ramped to 100% EtOAc for 30-45 cv and then held at 100% EtOAc 45-50 cv) on a 4 g silica column to afford N4-(4-fluorobenzyl)benzene-1,2,4-triamine in 43% yield (10.0 mg, 0.043 mmol) as yellow solid.

Description of reaction shown in FIG. 48, Panel D: To an NMR tube containing N4-(4-fluorobenzyl)benzene-1,2,4-triamine (10.0 mg, 0.043 mmol), triethylamine (6 μL, 0.043 mmol), and dry DCM (1 mL) was slowly added ethyl chloroformate (5 μL, 0.043 mmol) with sonication at room temperature. The resulting reaction mixture was stirred at room temperature for 2 h. After completion of reaction, it was diluted with DCM (2 mL) and washed with water (1 mL). The organic layer was dried over anhydrous MgSO₄, and the solvent was removed under vacuum to yield full conversion to the product.

Thus, in accordance with the present disclosure, there have been provided compositions, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
Ahrens et al. Chem. Rev. (2015) 115:931.
Amii et al. Chem. Rev. (2009) 109:2119.
Baird et al. Organometallics (2017) 36:1436.
Boogaerts et al. Eur. J. Med. Chem. (2010) 132:8858-8859.
Boogaerts et al. ACIE (2010) 49:8674-8677.
Borukhova et al. Org. Proc. Res. & Dev. (2016) 20:568-573.
Boyd et al. Bioorg. Med. Chem. Lett. (2000) 10:2557-2564.
Cantillo et al. Org. Lett. (2014) 16:896.
Chai et al. Eur. J. Med. Chem. (2009) 44:1913-1920.
Champagne et al. Synthesis (2015) 47:306.
Dalvit et al. Chem. Eur. J. (2014) 20:11058.
Den et al. The Journal of Chemical Physics (2014) 141:194303.
Ebert et al. J. Org. Chem. (2005) 70:4314-4317.
Faming Zhuanli Shenqing (Jun. 16, 2017) 106854136.
Fischer et al. Organometallics (2012) 31:1374-1383.
Hansch et al. Chem. Rev. (1991) 91:165.
Hernandez-Perez et al. Acc. Chem. Res. (2016) 49:1557.
Hossain et al. Angew. Chem. Int. Ed. (2002) 41:2335.
Hossain et al. J. Am. Chem. Soc. (2012) 134:11892.
Huaxi Yaoxue Zazhi (2005) 20(3):241-242.
Hutchby et al. Angew. Chem. Int. Ed. (2012) 51:548.
Khaled et al. J. Am. Chem. Soc. (2017) 139:13092.
Kiplinger et al. Chem. Rev. (1994) 94:373.
Konovalov et al. J. Phys. Chem. A (2000) 104:352.
Laev et al. European Journal of Organic Chemistry (2007) 2007:306.
Lentz et al. Chem. Int. Ed. Engl. (2013) 52:3328.
Leyva-Perez et al. ACS Catal. (2014) 4:722-731.
Lu et al. J. Am. Chem. Soc. (2016) 138:15805.
Mason et al. J. Am. Chem. Soc. (2000) 122:1814.
McDaniel et al. The Journal of Organic Chemistry (1958) 23:420.
McTeague et al. Angewandte Chemie International Edition (2016) 55:15072.
Molla et al. Green Chem. (2016) 18:4649-4656.
Mongin et al. Eur. J. Org. Chem. (2001) 2001(14):2771-2777.
Meyers et al. ACS Med. Chem. Lett. (2014) 5:89.
Olah et al. J. Org. Chem. (1979) 44:3872-3881.
Pastorekova et al. J. Enz. Inhib. Med. Chem. (2005) 20:211-217.
PCT App No. WO 2003/076405; Bayer Healthcare Ag, Applicant; published Sep. 18, 2003.
PCT App No. WO 2010/011812; Glaxosmithkline LLC et al., Applicant; published Jan. 28, 2010.
PCT App No. WO 2011/089126; Glaxo Group Limited et al., Applicant; published Jul. 28, 2011.
PCT App No. WO 2011/154700; Univ. St. Andrews et al., Applicant; published Dec. 15, 2011.
PCT App No. WO 2012/119046; Bioenergenix et al., Applicant; published Sep. 7, 2012.
Prakash et al. Adv. Synth. Catal. (2012) 354:2163-2171.
Prier et al. Chem. Rev. (2013) 113:5322.
Rackl et al. Green Chem. (2016) 18:214.
Savile et al. Science (2010) 329:305-309.
Schneider et al. Chem. Soc. (2012) 3:1381.
Senaweera et al. J. Am. Chem. Soc. (2014) 136:3002.
Senaweera et al. J. Am. Chem. Soc. (2016) 138:2520.
Senaweera et al. J. Org. Chem. (2014) 79:10466.
Shchegoleva et al. Chem Phys Lett (1999) 312:325.
Singh et al. Chem. Sci. (2015) 6:7206.
Singh et al. Chem. Sci. (2016) 7:6796.
Singh et al. J. Organomet. Chem. (2015) 776:51.
Sobkowski et al. Nucleosides Nucleotides Nucleic Acids (2010) 29:628.
Straathof et al. Angew. Chem. Int. Ed. (2016) 55:15549.
Staveness et al. Acc. Chem. Res. (2016) 49:2295.
Taylor et al. J. Amer. Chem. Soc. (2017) 139:8267-8276.
U.S. Pat. No. 4,404,216; Kenneth Richardson, inventor; issued Sep. 13, 1983.
Wang et al. Chem. Rev. (2014) 114:2432-2506.
Wang et al. J. Amer. Chem. Soc. (2015) 137:4626-4629.
Weaver et al. Tetrahedron (2014) 70:7413.
Xie et al. Angewandte Chemie International Edition (2016) 10.1002/anie.201602347, n/a.
Zhan et al. J. Phys. Chem. (2004) 108:2020.
Ziccarelli et al. Chem. Commun. (2016) 52:12729-12732.

What is claimed is:

1-24. (canceled)

25. A method of synthesizing a compound comprising a fluorinated aryl group utilizing hydrogen bond directed photocatalytic hydrodefluorination, the method comprising the steps of:
reacting perhalogenated benzoyl chloride with an aminobenzonitrile, resulting in a compound comprising a perhalogenated aryl group directly attached to a directing group comprising an acidic proton; and
reacting the compound comprising the perhalogenated aryl group and the directing group with a photocatalyst to regioselectively remove at least one halogen therefrom, thereby providing the compound comprising a fluorinated aryl group, and wherein at least one halogen is regioselectively removed from at least one of an ortho-position and a meta-position.

26. The method of claim 25, wherein the perhalogenated benzoyl chloride is 3-chloro-tetrafluorobenzoyl chloride.

27. The method of claim 26, wherein the chlorine at the 3-position and a fluorine at the 6-position are removed.

28-41. (canceled)

42. A method of synthesizing a compound comprising a fluorinated aryl group utilizing hydrogen bond directed photocatalytic hydrodefluorination, the method comprising the steps of:
reacting a perhalogenated arene with a directing group to install the directing group on the perhalogated arene, resulting in a compound comprising a perhalogenated aryl group directly attached to a directing group comprising an acidic proton; and
reacting the compound comprising the perhalogenated aryl group and the directing group with a photocatalyst to regioselectively remove at least one halogen therefrom, thereby providing the compound comprising a fluorinated aryl group, wherein the compound comprising the fluorinated aryl group is synthesized in at least about 50% yield without any chromatography steps.

43. The method of claim 42, wherein the perhalogenated arene is perhalogenated benzoyl chloride, the directing group is an aminobenzonitrile, and at least the halogens at a 3-position and a 6-position are removed.

44. The method of claim 42, wherein the perhalogated arene is a perhalogenated benzoic acid, and wherein the directing group is an aminobenzonitrile.

45. The method of claim 44, wherein at least the two halogens at meta-positions and at least one halogen at an ortho-position are removed.

46. The method of claim 44, wherein the two halogens at ortho-positions and the two halogens at meta-positions are removed.

47. The method of claim 42, wherein the perhalogated arene is a perhalogenated benzoic acid, wherein the directing group is an aminodihydrobenzoimidazolone, and wherein the two halogens at ortho-positions and the two halogens at meta-positions are removed.

48. A method of synthesizing a compound comprising a fluorinated aryl group utilizing hydrogen bond directed photocatalytic hydrodefluorination, the method comprising the steps of:
reacting perhalogenated benzoic acid with a directing group, resulting in a compound comprising a perhalogenated aryl group directly attached to a directing group comprising an acidic proton, wherein the perhalogenated benzoic acid is 3,5-dichloro-2,4,6-trifluorobenzoic acid; and
reacting the compound comprising the perhalogenated aryl group and the directing group with a photocatalyst to regioselectively remove at least one halogen therefrom, thereby providing the compound comprising a fluorinated aryl group, and wherein at least one halogen is regioselectively removed from at least one of an ortho-position and a meta-position.

49. The method of claim 48, wherein the directing group is an aminobenzonitrile.

50. The method of claim 49, wherein the two chlorines and a fluorine at an ortho-position are removed.

51. The method of claim 49, wherein the two chlorines and each of the fluorines at ortho-positions are removed.

52. The method of claim 49, wherein the directing group is a aminodihydrobenzoimidazolone, and wherein the two chlorines and each of the fluorines at ortho-positions are removed.
